SEQUENCE LISTING (43 sequences)

SEQ ID NO: 1 - protein, 13 aa, Ebola virus
GMVSLHVPEGETT

SEQ ID NO: 2 - protein, 13 aa, Ebola virus
LHVPEGETTLPSQ

SEQ ID NO: 3 - protein, 13 aa, Ebola virus
GMVPLHIPEGETT

SEQ ID NO: 4 - protein, 13 aa, Ebola virus
LHIPEGETTLPSQ

SEQ ID NO: 5 - protein, 117 aa, Artificial Sequence
  16F6 Heavy Chain
EVQLVESGGGLVTPGGSLKLSCAASGFAFNYYDMFWVRQNTEKRLEWVAYINSGGGNTYYPDTVKGRFTISRDNAKKTLFLQMSSLRSEDTAMYYCARQLYGNSFFDYWGQGTSLTV

SEQ ID NO: 6 - protein, 106 aa, Artificial Sequence
  16F6 Light Chain
DIVMTQSHKFMSTSVGDRVTITCKASQDVTTAVAWYQQKPGHSPKLLIYWASTRHTGVPDRFTGSGSGTDFTLTLNSVQAEDLALYYCQQHYSTPLTFGAGTKLEL

SEQ ID NO: 7 - protein, 154 aa, Artificial Sequence
  19F10 Heavy Chain
  misc_feature (148)..(149): Xaa can be any naturally occurring amino acid
MEWSWVFLFLLSGTAGVLSEVQLQQSGPELVKPGASVKISCKASGYSFTGYYMHWVKQSHVKSLEWIGRINPYNGATSYNQNFKDKASLTVDKSSSTAYMELHSLTSEDSAVYYCARPDYTSSFAYWGQGTLVTVSAAKTTPPPVYPXXPGSLG

SEQ ID NO: 8 - protein, 144 aa, Artificial Sequence
  19F10 Light Chain
MVSSAQFLGLLLLCFQGTRCDIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKRADAAPTVSIFSPSSKL

SEQ ID NO: 9 - protein, 150 aa, Artificial Sequence
  5G2 Heavy Chain
  misc_feature (4)..(4): Xaa can be any naturally occurring amino acid
  misc_feature (145)..(145): Xaa can be any naturally occurring amino acid
MEWXWVILFLMAVVTGVNSEVQLQQSGAELVKPGASVKLSCTASGFNIKDTYIHWVKQRPEEGLEWIGRIDPANDNIKFDPKFQGKATITADTSSNTAYLQLLSLTSEDTAVYFCARTNGLDYWGQTTLTVSSAKTTPPSVYPLXPGKAW

SEQ ID NO: 10 - protein, 149 aa, Artificial Sequence
  5G2 Light Chain
MKLPVRLLVLMFWIPASSSDVVMTQTPLSLPVSLGDQASISCRSSQSLLHSNGNTYSHWYLQKPGQSPKLLIYKVSNRLSGVPDRFSGSGSGTDFTLKISRVEAEDLGVYFCSQSTHVPYTFGGGTKLEIKRADAAPTVSIFPPSSKLG

SEQ ID NO: 11 - protein, 153 aa, Artificial Sequence
  17F6 Heavy Chain
MMVLSLLYLLTVVPGILSDVQLQESGPGLVKPSQTVSLTCTVTGISITAGNYRWSWIRQFPGNKLEWIGYIYYSGTIAYNPSLTSRTAITRDSSKNQFFLEMNSLTAEDTATYYCARDRGWLLLDYWGQGTTLTVSSAKTTAPSVYPLAPGSL

SEQ ID NO: 12 - protein, 148 aa, Artificial Sequence
  17F6 Light Chain
MKLPVRLLVLMFWIPASSSDVLMTQTPLSLPVSLGDQASISCRSSQTIVHSNGNTYLEWYLQKPGQSPKLLIYKVSSRFSGVPDRFSGSGSGTDFTLKISRVEAEDLGVYYCFQGSHFPYTFGGGTKLEIKRADAAPTVSIFPPSSKL

SEQ ID NO: 13 - protein, 120 aa, Artificial Sequence
  X10H2 Heavy Chain
QVQLEQSGAEVKKPGTSVKVSCKASGFTFGSDAISWVRQAPGQGLEWMGVIIPVVGARTYAEKFQGRITITADTSTSTVFMDLSSIRSDDTAVYYCAREGARAATGHYKSMDIWGRGTLV

SEQ ID NO: 14 - protein, 107 aa, Artificial Sequence
  X10H2 Light Chain
EIELTQSPSSLYASVGDKVTITCRASQGISTYLNWYQQKPGKAPKLLIYAASSLQSGVPSRFSGSGSGTEFTLTISSLQPEDFAAVYCQQYNSYPRTFGQGTKVEIK

SEQ ID NO: 15 - protein, 117 aa, Artificial Sequence
  X10B1 Heavy Chain
EVQLLESGPSLVKPSETLTLTCAVSGASISGDYLNWIRQPPGKGLEWIGGIFGSGGSTDYSPSFKSRVTISTDTSKNQFSLKLTSMTTADTAVYFCATALRGARFDAFDFWGQGRRV

SEQ ID NO: 16 - protein, 106 aa, Artificial Sequence
  X10B1 Light Chain
DIELTQSPATLSLSPGERATLSCRASQSVSTYLAWYQKKPGQAPRLLFYGASNRATGIPDRFSGRRSGTDFTLTISSLEPEDVGVYYCQQYNKWNSFGQGTKVEIK

SEQ ID NO: 17 - protein, 122 aa, Artificial Sequence
  X10F3 Heavy Chain
QVQLEQSGAEVKKPGTSVKVSCKASGFFTFGSDAISWVRQAPGQGLEWMGVIIPVVGARTYAEKFQGRITITADTSTSTVFMDLSSLRSDDTAVYYCAREGARAATGHYKSMDIWGRGRTLV

SEQ ID NO: 18 - protein, 107 aa, Artificial Sequence
  X10F3 Light Chain
ELQMTQSPSSLSASVGDRVTITCRASQDIGPFLNWYQHKPGKPPKLLIYDVSNLQDGVPSTFSGSGSGTDFTLTISNLQPEDFATYYCLHYANYPRTFGPGTKVEIK

SEQ ID NO: 19 - protein, 122 aa, Artificial Sequence; identical to SEQ ID NO: 17
  X10B6 Heavy Chain

SEQ ID NO: 20 - protein, 107 aa, Artificial Sequence
  X10B6 Light Chain
ELLMTQSPSSLSASVGDRVTITCRASQGISTYLSWFQQKPGKPPKLLIYLTSSLEDGVPSRFSGAGSGTEFTLTISNLQPEDFASYYCLQYQSYPRTFGPGTKVEIK

SEQ ID NO: 21 - protein, 330 aa, Artificial Sequence
  hlgG1 CH
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 107 aa, Artificial Sequence [sequence omitted]

SEQ ID NO: 23 - protein, 249 aa, Artificial Sequence
  Whole scFv X10H2
QVQLEQSGAEVKKPGTSVKVSCKASGFTFGSDAISWVRQAPGQGLEWMGVIIPVVGARTYAEKFQGRITITADTSTSTVFMDLSSLRSDDTAVYYCAREGARAATGHYKSMDIWGRGTLVTVSSASTKGPKLEEGEFSEARVEIELTQSPSSLYASVGDKVTITCRASQGISTYLNWYQQKPGKAPKLLIYAASSLQSGVPSRFSGSGSGTEFTLTISSLQPEDFAAYYCQQYNSYPRTFGQGTKVEIK

SEQ ID NO: 24 - protein, 6 aa, Artificial Sequence
  X10H2 CDR
QGISTY

SEQ ID NO: 25 - protein, 3 aa, Artificial Sequence
  X10H2 CDR
AAS

SEQ ID NO: 26 - protein, 9 aa, Artificial Sequence
  X10H2 CDR
QQYNSYPRT

SEQ ID NO: 27 - protein, 8 aa, Artificial Sequence
  X10H2 CDR
GFTFGSDA

SEQ ID NO: 28 - protein, 8 aa, Artificial Sequence
  X10H2 CDR
IIPVVGAR

SEQ ID NO: 29 - protein, 13 aa, Artificial Sequence
  X10H2 CDR
ARAATGHYKSMDI

SEQ ID NO: 30 - protein, 10 aa, Artificial Sequence
  16F6 CDR
GFAFNYYDMF

SEQ ID NO: 31 - protein, 15 aa, Artificial Sequence
  16F6 CDR
YINSGGGNTYYPDTV

SEQ ID NO: 32 - protein, 10 aa, Artificial Sequence
  16F6 CDR
QLYGNSFFDY

SEQ ID NO: 33 - protein, 5 aa, Artificial Sequence
  16F6 CDR
DVTTA

SEQ ID NO: 34 - protein, 5 aa, Artificial Sequence
  16F6 CDR
WASTR

SEQ ID NO: 35 - protein, 10 aa, Artificial Sequence
  16F6 CDR
CQQHYSTPLT

SEQ ID NO: 36 - protein, 119 aa, Artificial Sequence
  F4 Heavy Chain variable region
EVQLVESGGGLVQPGGSLRLSCAASGFAFNYYDMFWVRQAPGKGLEWVAYIKPGGGNTYYADSVKGRFTISADTSKNTAYLQMNSLRAEDTAVYYCARQLYGNSFFDYWGQGTLVTVSS

SEQ ID NO: 37 - protein, 107 aa, Artificial Sequence
  F4 Light Chain variable region
DIQMTQSPSSLSASVGDRVTITCKASQDVTTAVAWYQQKPGKAPKLLIYWASTRHTGVPSRFSGSGSGTDFTLTISSLQPEDFATYYCQQHYSTPLTFGQGTKVEIK

SEQ ID NO: 38 - protein, 10 aa, Artificial Sequence
  F4 CDR
GFAFNYYDMF

SEQ ID NO: 39 - protein, 15 aa, Artificial Sequence
  F4 CDR
YIKPGGGNTYYADSV

SEQ ID NO: 40 - protein, 10 aa, Artificial Sequence
  F4 CDR
QLYGNSFFDY

SEQ ID NO: 41 - protein, 5 aa, Artificial Sequence
  F4 CDR
DVTTA

SEQ ID NO: 42 - protein, 5 aa, Artificial Sequence
  F4 CDR
WASTR

SEQ ID NO: 43 - protein, 10 aa, Artificial Sequence
  F4 CDR
CQQHYSTPLT

(19) United States
(12) Patent Application Publication
(10) Pub. No.: US 2023/0174627 A1
(43) Pub. Date: Jun. 8, 2023

(54) ANTIBODIES TO SUDAN AND EBOLA VIRUS

(71) Applicants: Jeffrey W. FROUDE, Walkersville, MD (US); John M. DYE, Frederick, MD (US); Andrew S. HERBERT, Frederick, MD (US); Philippe THULLIER, Frederick, MD (US)

(72) Inventors: Jeffrey W. FROUDE, Walkersville, MD (US); John M. DYE, Frederick, MD (US); Andrew S. HERBERT, Frederick, MD (US); Philippe THULLIER, Frederick, MD (US)

(21) Appl. No.: 17/611,391

(22) PCT Filed: May 14, 2020
(86) PCT No.: PCT/US2020/032882

§ 371 Date: Nov. 15, 2021

Related U.S. Application Data
(60) Provisional application No. 62/847,451, filed on May 14, 2019.

(51) Int. Cl. C07K 16/10; A61P 31/14

(52) U.S. Cl. CPC C07K 16/10; A61P 31/14; C07K 2317/622; C07K 2317/14; C07K 2317/76; C07K 2317/24; A61K 2039/507

(57) ABSTRACT

Formulations and methods of treatment for a patient infected with Sudan vims are provided. The formulation comprises a therapeutically effective combination of: (i). a first monoclonal antibody comprising a heavy chain variable region comprising an amino acid sequence deduced from the nucleic acid molecule as set forth in SEQ. ID NO: 5, and a light chain variable region comprising an amino acid sequence deduced from the nucleic acid molecule as set forth in SEQ ID NO: 6, therapeutically effective mutations, and humanized variants thereof, and humanized variants thereof; (ii). a second monoclonal antibody comprising a heavy chain variable region comprising an amino acid sequence deduced from the nucleic acid molecule as set forth in SEQ. ID NO: 13 and a light chain variable region comprising an amino acid sequence deduced from the nucleic acid molecule as set forth in SEQ ID NO: 14, therapeutically effective mutations, and humanized variants thereof.

### STATEMENT AS TO RIGHTS OR INVENTIONS MADE UNDER FEDERALLY SPONSORED RESEARCH AND DEVELOPMENT

This invention was made with government support from the United States Army Medical Research Institute of Infectious Disease, an organization of the United States Army Medical Research and Materiel Command. The United States Government has certain rights in the invention.

### BACKGROUND

The 2013-2016 Ebola virus outbreak in western Africa clearly illustrated the devastating impact ebolaviruses can have on human health. The Ebolavirus genus, of the Filoviridae family, consists of five antigenically distinct species: Zaire ebolavirus (type member Ebola virus), Sudan ebolavirus (Sudan virus), Bundibugyo ebolavirus (Bundibugyo virus), Täi Forest ebolavirus, and Reston ebolavirus (1). Although Ebola virus (EBOV) caused the 2013-2016 outbreak that developed into the largest ebolavirus epidemic in recorded history (2), a related but antigenically-divergent virus, Sudan virus (SUDV), also presents a significant health threat. Since its discovery in 1976, SUDV has caused 8 confirmed outbreaks in equatorial Africa and infected 779 people, 412 of whom died (case-fatality rate 53%) (3).

Several vaccines and therapeutics are in advanced stages of development (4-9). Until recently, many ignored post-exposure antibody immunotherapy for the treatment of ebolavirus disease (EVD) due to several failed attempts to protect nonhuman primates (NHPs) from EBOV challenge (10, 11). Dye et al. were the first to report protection of macaques against EBOV infection using antibody-based immunotherapies (12). Subsequently, several independent groups reported on the post-exposure efficacy of monoclonal antibody (mAb)-based immunotherapies against EVD in macaques when administered as formulations or compositions of two or more mAbs (13-16). These landmark studies convincingly showed that combination mAb-based immunotherapies are a viable treatment option for EVD and galvanized therapeutic antibody development for filoviruses. ZMapp™, a formulation of three EBOV glycoprotein (GP)-specific mAbs, is currently the lead antibody-based therapy having advanced furthest down the licensure pathway (5). ZMapp™ is the only therapy tested in a randomized controlled trial during the West Africa outbreak (17) and is currently considered the standard of care by the Food and Drug Administration.

A number of large-scale antibody manufacturing methods are available, chief among them being mammalian cell-based expression systems (18). With the introduction of transgenic plants capable of glycosylating with mammalian N-glycans, and recent advancements in plant expression technology, plant-based manufacturing has the potential to become an alternative manufacturing system (19, 20). Ebolavirus-specific mAb material, utilized in the above-mentioned studies, was derived from various sources, including plants and mammalian cells. Pros and cons of each production method are still a topic of debate, but deliberations typically focus on aspects of regulatory affairs, safety, scalability of production, or immunogenicity, rarely on antibody performance. Previously reported evaluations of ebolavirus antibody therapies employed mAbs produced using disparate platforms, including hybridoma, mammalian cell, and plant production, making direct comparisons challenging.

An urgent needs exists for protection against SUDV. Described herein is the development of a protective a SUDV-specific mAb formulation while directly comparing plant- and mammalian-produced mAbs.

### SUMMARY

Compositions are provided for the treatment of Sudan virus. The composition comprises a therapeutically effective combination of: (i). a first monoclonal antibody comprising a heavy chain variable region comprising an amino acid sequence deduced from the nucleic acid molecule as set forth in SEQ. ID NO: 5, and a light chain variable region comprising an amino acid sequence deduced from the nucleic acid molecule as set forth in SEQ ID NO: 6, therapeutically effective mutations, and humanized variants thereof, and therapeutically effective mutations, and humanized variants thereof; and (ii). a second monoclonal antibody comprising a heavy chain variable region comprising an amino acid sequence deduced from the nucleic acid molecule as set forth in SEQ. ID NO: 13 and a light chain variable region comprising an amino acid sequence deduced from the nucleic acid molecule as set forth in SEQ ID NO: 14, therapeutically effective mutations, and humanized variants thereof, therapeutically effective mutations, and humanized variants thereof. The composition may further comprise a pharmaceutically acceptable excipient or carrier.

Methods are provided for treatment of Sudan virus infection in a patient. First, a patient in need of Sudan virus treatment is identified. A therapeutically effective amount of the composition is administered to the patient. The composition comprises a combination of (i) a first monoclonal antibody comprising a heavy chain variable region comprising an amino acid sequence deduced from the nucleic acid molecule as set forth in SEQ. ID NO: 5, and a light chain variable region comprising an amino acid sequence deduced from the nucleic acid molecule as set forth in SEQ ID NO: 6, therapeutically effective mutations, and humanized variants thereof, and therapeutically effective mutations, and humanized variants and a (ii) a second monoclonal antibody comprising a heavy chain variable region comprising an amino acid sequence deduced from the nucleic acid molecule as set forth in SEQ. ID NO: 13 and a light chain variable region comprising an amino acid sequence deduced from the nucleic acid molecule as set forth in SEQ ID NO: 14, therapeutically effective mutations, and humanized variants thereof, therapeutically effective mutations, and humanized variants thereof. The therapeutically effective composition may further comprise a pharmaceutically acceptable excipient or carrier.

According to another embodiment, disclosed is a monoclonal antibody or its antigen-binding fragments thereof, wherein the light chain variable region (V_{L}) of the monoclonal antibody comprises CDRs with the amino acid sequences of SEQ ID NOs: 24, 25 and 26; and/or wherein the heavy chain variable region (VH) of the monoclonal antibody comprises CDRs with the amino acid sequences of SEQ ID NOs: 27, 28, and 29. The monoclonal antibody, or its antigen-binding fragments thereof, bind to X10H2. In a specific example, the monoclonal antibody or its antigen-binding fragments thereof includes a V_{L} having the amino acid sequence of SEQ ID NO: 14; and/or a V_{H} having the amino acid sequence of SEQ ID NO: 13. The sequences or part of the sequences may be contained in Fab, Fab′, F(ab′)₂, Fv, CDR fragments, single-chain antibodies (e.g. scFv), humanized antibodies, chimeric antibodies, or bispecific antibodies.

Another embodiment disclosed is a method of treating Sudan virus infection in a patient involving administering to the patient a therapeutically effective amount of a composition comprising a monoclonal antibody, or its antigen-binding fragments as described above.

### BRIEF DESCRIPTION OF THE DRAWINGS

The following drawings form part of the present specification and are included to further demonstrate certain embodiments of the present invention. The invention may be better understood by reference to one or more of these drawings in combination with the detailed description of specific embodiments presented herein.

FIG. 1A-1C. Antigen specificity and affinity of humanized SUDV-specific mAbs. FIG. 1A Mammalian-derived (blue) and plant-derived (green) mAbs were serially diluted and added to plates coated with rVSV-SUDV-Bon GP. Following incubation with anti-human IgG-HRP, ABTS substrate was added and absorbance was measured. Embedded table reports the total Area Under Curve calculated for each mAb. Data points represent the mean±standard error of the mean (SEM) of two replicate assays, each having two technical replicates. FIG. 1B Competitive binding of humanized SUDV-specific mAbs. ELISAs were completed to identify mAbs that compete for SUDV GP binding. Chimeric (c) human version of indicated mAbs were added to antigen coated plates prior to the addition of murine (m) version of indicated mAbs. Murine antibody binding was detected using anti-mouse IgG-HRP secondary antibody. Data is presented as the percent of murine antibody alone for each mAb. FIG. 1C Antibody affinities for recombinant SUDV-Bon GP were determined using bilayer interferometry on the Octet® platform. AHC biosensors coated with plant or mammalian mAbs were sequentially dipped into assay buffer containing recombinant SUDV-Bon GP to measure GP on rate (kₒₙ), then into assay buffer alone to measure GP off rate (k_{off}). Affinity (K_{D}) was calculated using ForteBio data analysis software. Data represents the mean of two replicate assays, each having two technical replicates.

FIG. 2A-2C. In vitro characterization of SUDV-specific mAbs. FIG. 2A rVSV-SUDV GP escape mutants to X10H2 were selected by growing the virus in the presence of IC90 concentrations of X10H2. Escape variants were then sequenced to identify GP mutations. Neutralizing activity of X10H2, X10B6 and X10F3 against wild-type rVSV-SUDV GP (GP_{WT}) and escape mutant rVSV-SUDV GP (GPₘᵤₜ) was evaluated. Neutralizing activity of X10H2 against cleaved rVSV-SUDV GP (GP_{CL}) was also evaluated. Data points represent the mean±standard error of the mean (SEM) of two replicate assays, each having two technical replicates. FIG. 2B SUDV GP structure with labeled subunits, functional elements, and known antibody epitopes. FIG. 2C Neutralizing activity of plant (p, dotted line) and mammalian (m, solid line) mAbs were evaluated by microneutralization assay. Serially diluted mAbs were mixed with SUDV prior to adding to Vero cells at an MOI of 0.5. Cells were fixed 48 hours after infection and immunostained with SUDV-specific antibody and fluorescently labeled secondary antibody. Cells were imaged and the percent of infected cells determined using an Operetta and Harmonia Software. Data is presented as percent inhibition relative to untreated, infected control cells and represents the mean±SEM of two replicate assays, each having two technical replicates.

FIG. 3A-3D. Line graphs that illustrate in vivo efficacy of SUDV-specific mAbs in rodent model of SUDV disease. (FIG. 3A to FIG. 3B) Protective efficacy of mammalian and plant-derived SUDV-specific mAbs were compared directly in a weight loss mouse model of SUDV disease. Groups (N=10) of IFNAR−/− mice (5-8 weeks old) were infected with 1000 plaque forming units (pfu) of SUDV and treated intraperitoneally (I.P.) with 200 μg of FIG. 3A base binding mAbs or FIG. 3B mucin/cap binding mAbs on days 1 and 4 post-exposure. Vehicle control mice were treated I.P. with an equal volume of PBS on days 1 and 4 post-exposure. Percent weight change was calculated using the daily average mouse weight (group weight/N) relative to starting average mouse weight. (FIG. 3C to FIG. 3D) SUDV-specific mAbs from each competition group were down-selected using a lethal mouse model of SUDV disease. Groups (N=10 per study) of IFNAR−/− mice (4 weeks old) were infected with 1000 pfu of SUDV and treated I.P. with indicated mAb on days 1 and 4 post-exposure. (FIG. 3C) Percent weight change was calculated using the daily average mouse weight (group weight/N) relative to starting average mouse weight and data points represent the mean±standard error of the mean of two replicate studies (except X10B6). (FIG. 3D) Overall percent survival calculated using survival data from both studies combined (N=20 mice per group except X10B6 where N=10). (**P<0.01, ***P<0.001)

FIG. 4A-4E. In vivo efficacy of SUDV-specific mAbs in macaque model of SUDV disease. Rhesus macaques were exposed intramuscularly to 1750 plaque forming units of SUDV. Groups (N=4) were treated intravenously with either 50 mg/kg of SUDV-specific mAb formulation (composition) 16F6/X10H2 or and equal volume to weight ratio of vehicle (0.9% NaCl) on days 4 and 6 post-exposure (Ctrl 3 and Ctrl 4 treated on day 5 only). indicates mAb treatment days. (FIG. 4A to FIG. 4B) Macaques were monitored daily for (FIG. 4A) survival and (FIG. 4B) clinical signs of disease. (FIG. 4C to FIG. 4E) Blood was collected on indicated days post-exposure to evaluate viremia by (FIG. 4C) RT-PCR (****P<0.0001) or (FIG. 4D) plaque assay (*P<0.05) and (FIG. 4E) SUDV GP-specific IgG titers by end titer ELISA (****P<0.0001). Individual data points represent the mean from two replicate assays, each having at least two technical replicates. Lines represent group means.

FIG. 5A-5D. Serum chemistries and complete blood counts (CBC) of rhesus macaques following SUDV exposure. Rhesus macaques were exposed intramuscularly to 1750 plaque forming units of SUDV. Groups (N=4) were treated intravenously with either 50 mg/kg of SUDV-specific mAb formulation (composition) 16F6/X10H2 or and equal volume to weight ratio of vehicle (0.9% NaCl) on days 4 and 6 post-exposure (Ctrl 3 and Ctrl 4 treated on day 5 only). (FIG. 5A to FIG. 5D) Serum and whole blood was collected on indicated days post-exposure to evaluate serum enzymes (FIG. 5A) ALT, (FIG. 5B) AST, and (FIG. 5C) ALP by Piccolo and (FIG. 5D) platelet counts by CBC. Individual data points are from a single assay using instruments validated daily with appropriate instrument controls. Lines represent group means. *P<0.05, **P<0.01, ****P<0.0001

FIG. 6. An illustration that show the chimerization strategy for humanized SUDV-specific mAbs. Variable heavy and variable light chains from SUDV-specific murine or macaque mAbs were cloned into human IgG1 constant heavy and constant light chain expression vectors were then co-expressed in either mammalian or plant production systems to generate human IgG1 chimeric mAbs with SUDV specificity.

FIG. 7A-7B are line graphs that illustrate cross-reactive specificity of humanized SUDV-specific mAbs. Mammalian-derived (●) and plant-derived (▪) mAbs were serially diluted and added to plates coated with either recombinant (A) EBOV GP or (B) BDBV GP. Following incubation with anti-human IgG-HRP, ABTS substrate was added and absorbance was measured. Binding profiles of mammalian and plant generated mAbs are compared directly for each antigen.

FIG. 8 is a photograph of a SPOTS membrane that illustrates linear epitope specificity of SUDV GP-specific mAb 17F6. SPOTS membrane, coated with 13 mer peptides of SUDV GP from Boniface and GuIu isolates, was sequentially incubated with 17F6, HRP conjugated secondary antibody, and substrate to identify epitope specificity of 17F6. Amino acid sequence: 88=GMVSLHVPEGETT (Boniface) (SEQ ID NO.: 1); 89=LHVPEGETTLPSQ (Boniface) (SEQ ID NO.: 2); 184=GMVPLHIPEGETT (GuIu) (SEQ ID NO.: 3); 185-LHIPEGETTLPSQ (GuIu) (SEQ ID NO.: 4).

FIG. 9A-H are line graphs that illustrate weights, temperatures, and serum chemistries of rhesus macaques following SUDV exposure. Rhesus macaques, exposed intramuscularly to SUDV, were treated with SUDV-specific mAb formulation (composition) 16F6/X10H2 (light) or diluent alone (dark) on days 4 and 6 post-exposure. (A to B) Weights (A) and temperatures (B) were collected on indicated days post-exposure. Serum was collected on indicated days post-exposure to evaluate serum enzymes BUN (C), GGT (D), GLI (E), Ca (F), CRE (G), and AMY (H) by Piccolo.

In the Summary above, in the Detailed Description below, and the claims below, as well as the accompanying figures, reference is made to particular features of the invention. It is to be understood that the disclosure of the invention in this specification includes all possible combinations of such particular features. For example, where a particular feature is disclosed in the context of a particular embodiment or embodiment of the invention, or a particular claim, that feature can also be used, to the extent possible, in combination with and/or in the context of other particular embodiments and embodiments of the invention, and in the invention generally. For the purposes of explanation, numerous specific details are set forth in order to provide a thorough understanding of the present invention. It will be apparent, however, to one skilled in the art that the present invention may be practiced without these specific details.

### DETAILED DESCRIPTION

### 1. Definitions

As defined herein, “humanized antibodies” refer to antibodies with reduced immunogenicity in humans.

As defined herein, the word “treat” includes therapeutic treatment, where a condition to be treated is already known to be present and prophylaxis—i.e., prevention of, or amelioration of, the possible future onset of a condition.

As defined herein, a “therapeutically effective” treatment refers a treatment that is capable of producing a desired effect. Such effects include, but are not limited to, enhanced survival, reduction in presence or severity of symptoms, reduced time to recovery, and prevention of initial infection.

As defined herein, an “antibody” is meant to be a monoclonal antibody (mAb), or an immunologically effective fragment thereof, such as an Fab, Fab′, or F (ab′) 2 fragment. In some contexts, regardless of whether fragments are specified, the term “antibody” includes such fragments as well as single-chain forms. As long as the protein retains the ability specifically to bind its intended target, it is included within the term “antibody.” Also included within the definition “antibody” are single chain forms.

The basic antibody structural unit is known to comprise a tetramer. Each tetramer is composed of two identical pairs of polypeptide chains, each pair having one “light” chain (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Light chains are classified as kappa and lambda. Heavy chains are classified as gamma, mu, alpha, delta, or epsilon, and define the antibody's isotype as IgG, IgM, IgA, IgD and IgE, respectively. Within each isotype, there may be subtypes, such as IgGi, IgG₂, IgG₃, IgG₄, etc. Within light and heavy chains, the variable and constant regions are joined by a “J” region of about 12 or more amino acids, with the heavy chain also including a “D” region of about 3 or more amino acids. The particular identity of constant region, the isotype, or subtype does not impact the present invention. The variable regions of each light/heavy chain pair form the antibody binding site.

Two binding sites exist in an intact antibody The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4.

As used herein, the term “antigen binding fragments” refers to a polypeptide containing fragments of a full-length antibody, maintaining the ability to bind specifically to the same antigen, and/or to compete with the full length antibody to bind to the antigen, which is also called “the antigen binding portion”. See Fundamental Immunology, Ch. 7 (Paul, W., ed. 2, Raven Press, N.Y. (1989)), including the entire article and references in this invention for all purposes. Antigen binding fragments can be generated by recombinant DNA techniques or by cleaving intact antibodies with proteolytic enzymes or chemicals. In some cases, the antigen binding fragments include Fab, Fab′, F(ab)₂, Fd, Fv, dAb, and CDR fragments, single chain antibodies (e.g., scFv), chimeric antibodies, diabody, and the polypeptides that at least contains an antibody portion which is sufficient to confer a specific antigen binding capacity to the polypeptides.

A “humanized antibody,” as defined herein, is an antibody that is composed partially or fully of amino acid sequences derived from a human antibody germline by altering the sequence of an antibody having non-human complementarity determining regions (CDR). A humanized antibody does not encompass a chimeric antibody, having a mouse variable region and a human constant region. However, the variable region of the antibody and even the CDR are humanized by techniques that are by now well known in the art. The framework regions of the variable regions are substituted by the corresponding human framework regions leaving the non-human CDR substantially intact. As mentioned above, it is sufficient for use in the methods of the invention, to employ an immunologically specific fragment of the antibody, including fragments representing single chain forms.

Humanized antibodies have at least three potential advantages over non-human and chimeric antibodies for use in human therapy. First, because the effector portion is human, it may interact better with the other parts of the human immune system (e.g., destroy the target cells more efficiently by complement-dependent cytotoxicity (CDC) or antibody-dependent cellular cytotoxicity (ADCC)). Second, the human immune system should not recognize the framework or C region of the humanized antibody as foreign, and therefore the antibody response against such an injected antibody should be less than against a totally foreign non-human antibody or a partially foreign chimeric antibody. 3) Injected non-human antibodies have been reported to have a half-life in the human circulation much shorter than the half-life of human antibodies. Injected humanized antibodies will have a half-life essentially identical to naturally occurring human antibodies, allowing smaller and less frequent doses to be given.

The term “sequence identity” or “identity,” as used herein in the context of two polynucleotides or polypeptides, refers to the residues in the sequences of the two molecules that are the same when aligned for maximum correspondence over a specified comparison window. As used herein, the term “percentage of sequence identity” or “% sequence identity” refers to the value determined by comparing two optimally aligned sequences (e.g., nucleic acid sequences or polypeptide sequences) of a molecule over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa.

It is of note that in all embodiments describing preparation of humanized antibodies, these antibodies are screened to ensure that antigen binding has not been disrupted. This may be accomplished by any of a variety of means known in the art, but one convenient method would involve use of a phage display library. As will be appreciated by one of skill in the art, as used herein, ‘immunoreactive fragment’ refers in this context to an antibody fragment reduced in length compared to the wild-type or parent antibody which retains an acceptable degree or percentage of binding activity to the target antigen. As will be appreciated by one of skill in the art, what is an acceptable degree will depend on the intended use.

Other sequences are possible for the light and heavy chains for the human or humanized antibodies of the present invention. The immunoglobulins can have two pairs of light chain/heavy chain complexes, at least one chain comprising one or more mouse complementarity determining regions functionally joined to human framework region segments.

The polynucleotides will typically further include an expression control polynucleotide sequence operably linked to the humanized immunoglobulin coding sequences, including naturally-associated or heterologous promoter regions. Preferably, the expression control sequences will be eukaryotic promoter systems in vectors capable of transforming or transfecting eukaryotic host cells, but control sequences for prokaryotic hosts may also be used. Once the vector has been incorporated into the appropriate host cell line, the host cell is propagated under conditions suitable for expressing the nucleotide sequences, and, as desired, the collection and purification of the light chains, heavy chains, light/heavy chain dimers or intact antibodies, binding fragments or other immunoglobulin forms may follow. The nucleic acid sequences of the present invention capable of ultimately expressing the desired humanized antibodies can be formed from a variety of different polynucleotides (genomic or cDNA, RNA, synthetic oligonucleotides, etc.) and components (e.g., V, J, D, and C regions), using any of a variety of well-known techniques. Joining appropriate genomic and synthetic sequences is a common method of production, but cDNA sequences may also be utilized.

Human constant region DNA sequences can be isolated in accordance with well-known procedures from a variety of human cells, but preferably from immortalized B-cells. Suitable source cells for the polynucleotide sequences and host cells for immunoglobulin expression and secretion can be obtained from a number of sources well known in the art.

### 2. Formulations (Compositions)

Currently, no antibody-based therapies treat Sudan virus (SUDV) infection. Disclosed herein is a panel of SUDV glycoprotein (GP)-specific human chimeric monoclonal antibodies (mAbs) produced using both plant and mammalian expression systems that completed head-to-head in vitro and in vivo evaluations. Neutralizing activity, competitive binding groups, and epitope specificity of each mAb were defined before assessing protective efficacy of individual mAbs using a mouse model of SUDV infection. Of the mAbs tested, GP base-binding mAbs were more potent neutralizers and more protective than glycan cap- or mucin-like domain-binding mAbs. No significant performance difference was observed between plant and mammalian mAbs in any in vitro or in vivo evaluation.

Therefore, formulations (or compositions) are provided for the treatment of SUDV infection comprising a therapeutically effective combination of: (i). a first monoclonal antibody comprising a heavy chain variable region comprising an amino acid sequence deduced from the nucleic acid molecule as set forth in SEQ. ID NO: 5, and a light chain variable region comprising an amino acid sequence deduced from the nucleic acid molecule as set forth in SEQ ID NO: 6, therapeutically effective mutations, and humanized variants thereof, and humanized variants thereof; (ii). a second monoclonal antibody comprising a heavy chain variable region comprising an amino acid sequence deduced from the nucleic acid molecule as set forth in SEQ. ID NO: 13 and a light chain variable region comprising an amino acid sequence deduced from the nucleic acid molecule as set forth in SEQ ID NO: 14, therapeutically effective mutations, and humanized variants thereof. The formulations (or compositions) may further comprise a pharmaceutically acceptable excipient or carrier. Preferably, the formulation or composition is for the treatment of SUDV infection in a human.

Immunization of an appropriate host, or as described below, the identification of subjects who are immune due to prior natural infection. Antibody-producing cells may be induced to expand by priming with immunogens is the first step in generating monoclonal antibodies. A variety of routes can be used to administer such immunogen (subcutaneous, intramuscular, intradermal, intravenous and intraperitoneal). The production of antibodies may be monitored by sampling blood of the immunized animal at various points following immunization. A second, booster injection, also may be given. The process of boosting and titering is repeated until a suitable titer is achieved. When a desired level of immunogenicity is obtained, the animal can be bled and the serum isolated and stored, and/or the animal can be used to generate mAbs.

Somatic cells with the potential for producing antibodies, specifically B lymphocytes (B cells), are selected for use in the mAb generating protocol. These cells may be obtained from biopsied spleens or lymph nodes, or from circulating blood. The antibody-producing B lymphocytes from the immunized animal are then fused with ceils of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art.

For culturing, the selection medium may be HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain is also used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

It also is contemplated that a molecular cloning approach may be used to generate monoclonal antibodies. For this, RNA can be isolated from the hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

A head-to-head comparison of antibodies produced in plants and mammalian expression systems was performed. The comparison was limited to antibody performance, specifically antigen binding, virus neutralization, and in vivo protection. The in vitro assessment failed to illustrate any significant differences between the plant- and mammal-derived mAbs investigated. For individual mAbs, plant and mammalian products showed similar antigen binding kinetics and virus neutralization activity. Notably, the in vitro assays were a measurement of and dependent on Fab activity. None of the in vitro assays evaluated Fc function directly. The in vivo assessment also did not identify any significant differences between plant- and mammal-derived mAbs. Both performed equally well in protecting mice from SUDV disease. Significant differences in Fc function, if they exist, could potentially have been observed by in vivo testing, although the mouse model may not be suitable to highlight small differences in the Fc effector functions of plant and mammalian mAbs.

Antibodies according to the present disclosure may be defined, in the first instance, by their binding specificity which in this case is for Sudan virus glycoprotein (GP). Those of skill in the art, by assessing the binding specificity/affinity of a given antibody using techniques well known to those of skill in the art, can determine whether such antibodies fall within the scope of the instant claims. In one aspect, there are provided monoclonal antibodies having clone-paired CDRs from the heavy and light chains as illustrated in Table 1. Such antibodies may be produced by the clones discussed below in the Examples section using methods described herein.

The antibodies may be defined by their variable sequence that include additional “framework” regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions.

In various embodiments, one may choose to engineer sequences of the identified antibodies for a variety of reasons, such as improved expression, improved cross-reactivity or diminished off-target binding. The following is a general discussion of relevant techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full length IgG antibodies were generated by subcloning heavy and light chain Fv DNAs from the cloning vector into an IgG plasmid vector, transfected into 293 Freestyle cells or CHO cells, and antibodies were collected an purified from the 293 or CHO cell supernatant.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F (ab′), F (ab′) 2) that are produced, for example, by the proteolytic cleavage of the mAbs, or single-chain immunoglobulins producible, for example, via recombinant means. Such antibody derivatives are monovalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. It also is understood in the art that the substitution of like amino acids can be made effectively on the basis of hydrophilicity.

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

Modified antibodies may be made by any technique known to those of skill in the art, including expression through standard molecular biological techniques, or the chemical synthesis of polypeptides. Methods for recombinant expression are addressed elsewhere in this document.

In certain embodiments, the antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Monoclonal antibodies produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods that include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate PEG antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies is bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaidi et al, 1977). It will therefore be appreciated that under differing electrophoresis conditions, the apparent molecular weights of purified or partially purified expression products may vary. III. Active/Passive Immunization and Treatment/Prevention of Ebola Virus Infection The present disclosure provides pharmaceutical compositions comprising anti-ebolavirus antibodies and antigens for generating the same. Such compositions comprise a prophylactically or therapeutically effective amount of an antibody or a fragment thereof, or a peptide immunogen, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The formulation of the present invention, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Formulations of the present disclosure, as described herein, can be formulated as a vaccine for parenteral administration, e.g., formulated for injection via the intradermal, intravenous, intramuscular, subcutaneous, or even intraperitoneal routes. Administration by intradermal and intramuscular routes are contemplated. The vaccine could alternatively be administered by a topical route directly to the mucosa, for example by nasal drops, inhalation, or by nebulizer. Pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylarnine, 2-ethylamino ethanol, histidine, procaine, and the like.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of formulations of the disclosure are supplied either separately or mixed together in unit dosage form, for example, as a dry lyophilized powder or water-free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The formulations of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In addition to the humanized immunoglobulins specifically described herein, other “substantially homologous” modified immunoglobulins can be readily designed and manufactured utilizing various recombinant DNA techniques well known to those skilled in the art. For example, the framework regions can vary from the native sequences at the primary structure level by several amino acid substitutions, terminal and intermediate additions and deletions, and the like. Moreover, a variety of different human framework regions may be used singly or in combination as a basis for the humanized immunoglobulins of the present invention. In general, modifications of the genes may be readily accomplished by a variety of well-known techniques, such as site-directed mutagenesis.

Alternatively, polypeptide fragments comprising only a portion of the primary antibody structure may be produced, which fragments possess one or more immunoglobulin activities (e.g., complement fixation activity). These polypeptide fragments may be produced by proteolytic cleavage of intact antibodies by methods well known in the art, or by inserting stop codons at the desired locations in vectors using site-directed mutagenesis, such as after CHI to produce Fab fragments or after the hinge region to produce F(ab′)2 fragments. Single chain antibodies may be produced by joining NL and NH with a DNA linker. As stated previously, the polynucleotides will be expressed in hosts after the sequences have been operably linked to (i.e., positioned to ensure the functioning of) an expression control sequence. These expression vectors are typically replicable in the host organisms either as episomes or as an integral part of the host chromosomal DNA. Commonly, expression vectors will contain selection markers, e.g., tetracycline or neomycin, to permit detection of those cells transformed with the desired DNA sequences. Expression vectors for these cells can include expression control sequences, such as an origin of replication, a promoter, an enhancer, and necessary processing information sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, cytomegalovirus and the like. The vectors containing the polynucleotide sequences of interest (e.g., the heavy and light chain encoding sequences and expression control sequences) can be transferred into the host cell by well-known methods, which vary depending on the type of cellular host. A variety of hosts may be employed to express the antibodies of the present invention using techniques well known in the art. Mammalian tissue cell culture is preferred, especially using, for example, CHO, COS, Syrian Hamster Ovary, HeLa, myeloma, transformed B-cells, human embryonic kidney, or hybridoma cell lines.

It is of note that as discussed herein, any of the described antibodies or humanized variants thereof may be formulated into a pharmaceutical treatment for providing passive immunity for individuals suspected of or at risk of developing hemorrhagic fever comprising a therapeutically effective amount of said antibodies. The pharmaceutical preparation may include a suitable excipient or carrier. See, for example, Remington: The Science and Practice of Pharmacy, 1995, Gennaro ed. As will be apparent to one knowledgeable in the art, the total dosage will vary according to the weight, health and circumstances of the individual as well as the efficacy of the antibodies.

In a preferred embodiment, the antibodies of the present invention are produced in plant expression systems. Specifically, anti-SUDV GP IgG1 full light chain and heavy chain sequences were designed using variable domain amino acid sequences. Signal peptide derived from Nicotiana tabacum pathogenesis-related 1a (PR1a) protein (X06930) was fused with variable heavy and light chain regions to target expression of antibodies to the apoplast. The light chain variable domains of 16F6, X10B1, X10B6, X10F3 and X10H2 were fused to a human kappa constant region to produce full length chimeric light chains, while the heavy chain variable domains were fused to a gamma constant human IgG1 region to produce chimeric IgG1 heavy chains. The light chain variable domains of 19F10 and 5G2 were fused with a mouse constant region to produce full length mouse light chains, while the heavy chain variable domains were fused with a gamma constant mouse IgG1 region to produce chimeric IgG1 heavy chains. All genes were codon optimized to ensure high level expression in Nicotiana benthamiana plants and synthetized (ThermoFisher Scientific). All genes were then cloned into the binary expression vector pGR-D35S, where expression of heavy and light chains is under control of the double 35S Cauliflower mosaic virus (CaMV) promoter and Tobacco etch virus (TEV) enhancer. Agrobacterium tumefaciens strain AGL1 was transformed with the plasmids. N. benthamiana plants were vacuum infiltrated using agrobacterium cultures and aerial tissue was harvested after four to six days.

In other preferred embodiments, the antibodies of the present invention are produced in a mammalian expression system. A total of seven chimeric anti-SUDV GP full-length heavy chain and light chain sequences were designed using the variable domain amino acid sequences of 16F6, 17F6, 19F10, 5G2, X10B1, X10F3 and X10H2. All antibody sequences were constructed utilizing murine light and heavy chain signal peptides, human IgG1 heavy constant sequences and human kappa constant sequences. All genes were codon and sequence optimized to ensure high level expression in murine cells and synthetized (GeneArt/ThermoFisher Scientific). Stable NS0 cell lines were developed at BioFactura, Inc. (Frederick, Md.) using the StableFast platform and the single cholesterol selection strategy as described by Sampey, et. al. (36). Briefly, bicistronic expression plasmids were constructed coding for both heavy and light chains of each mAb. Each chain coding sequence was flanked upstream by cytomegalovirus (CMV)-derived promoters and downstream by bovine growth hormone (BGH) polyadenylation sequences (poly-A) comprising independent heavy and light chain expression cassettes. The cholesterol selection marker 17β-hydroxysteroid dehydrogenase type 7 or Hsd17β7 was regulated by an SV40 promoter and SV40 poly-A. The serum-free medium adapted cholesterol auxotrophic NS0 host cell line (NS0-SF, ECACC, Cat No. 03061601) was transfected by electroporation by either the expression plasmids and stable cell lines were selected by withdrawal of exogenous cholesterol. Best performing pools for each mAb were scaled to shaker or spinner flasks and stirred-tank single-use bioreactors (BioBLU, Eppendorf) and operated in fed-batch mode for 7-9 days. Cultures were subsequently clarified by centrifugation and filtration and mAbs were purified by a single Protein A capture step. Briefly, clarified supernatants were loaded onto Amsphere Protein A columns (JSR Life Sciences). Columns were washed with equilibration buffer (20 mM sodium phosphate, 150 mM sodium chloride) followed by a high salt wash with 500 mM sodium chloride. The mAbs were eluted with 0.2 M glycine at pH 3.0 directly into a container with 1 M Tris-HCl pH 8.0 to neutralize the eluate. Final purified mAbs were dialyzed into 1×PBS, pH 7.4 and sterile filtered. Final purified mAbs were characterized for identity, purity and endotoxin levels.

Consistent manufacturing and quality control of an antibody drug substance with predefined specifications is a key feature for the successful development of antibody mixtures for human therapeutic use [Zwick, M. B., et al. (2001). Bioeng 94(2): 396-405; Nielsen, L. S., et al. (2010).

Once expressed, the antibodies can be purified according to standard procedures. Substantially pure immunoglobulins of at least about 90 to 95% homogeneity are preferred, and 98 to 99% or more homogeneity most preferred, for pharmaceutical uses. Once purified, partially or to homogeneity as desired, the polypeptides may then be used therapeutically, as directed herein.

The concentration of the humanized antibody in formulations from as low as about 0.1% to as much as 15 or 20% by weight and will be selected primarily based on fluid volumes, viscosities, and so forth, in accordance with the particular mode of administration selected. Thus, a pharmaceutical composition for injection could be made up to contain in 1 mL of phosphate buffered saline from 1 to 100 mg of the humanized antibody of the present invention. The formulation could be sterile filtered after making the formulation, or otherwise made microbiologically acceptable. A typical composition for intravenous infusion could have a volume as much as 250 mL of fluid, such as sterile Ringer's solution, and 1-100 mg per mL, or more in antibody concentration. Therapeutic agents of the invention can be frozen or lyophilized for storage and reconstituted in a suitable sterile carrier prior to use. Lyophilization and reconstitution can lead to varying degrees of antibody activity loss (e.g. with conventional immune globulins, IgM antibodies tend to have greater activity loss than IgG antibodies). Dosages may have to be adjusted to compensate. The pH of the formulation will be selected to balance antibody stability (chemical and physical) and comfort to the patient when administered.

Generally, pH between 4 and 8 is tolerated. Although the foregoing methods appear the most convenient and most appropriate for administration of proteins such as humanized antibodies, by suitable adaptation, other techniques for administration, such as transdermal administration and oral administration may be employed provided proper formulation is designed. In addition, it may be desirable to employ controlled release formulations using biodegradable films and matrices, or osmotic mini-pumps, or delivery systems based on dextran beads, alginate, or collagen. In summary, formulations are available for administering the antibodies of the invention and are well-known in the art and may be chosen from a variety of options. Typical dosage levels can be optimized using standard clinical techniques and will be dependent on the mode of administration and the condition of the patient.

### 3. Methods of Treatment

Methods of treatment of SUDV disease are provided. A method of treating SUDV infection in a patient includes identifying a patient in need SUDV treatment; and administering to the patient a therapeutically effective amount of a formulation or a composition comprising a combination of: (i). a first monoclonal antibody comprising a heavy chain variable region comprising an amino acid sequence deduced from the nucleic acid molecule as set forth in SEQ. ID NO: 5, and a light chain variable region comprising an amino acid sequence deduced from the nucleic acid molecule as set forth in SEQ ID NO: 6, therapeutically effective mutations, and humanized variants thereof, and therapeutically effective mutations, and humanized variants thereof; (ii). a second monoclonal antibody comprising a heavy chain variable region comprising an amino acid sequence deduced from the nucleic acid molecule as set forth in SEQ. ID NO: 13 and a light chain variable region comprising an amino acid sequence deduced from the nucleic acid molecule as set forth in SEQ ID NO: 14, therapeutically effective mutations, and humanized variants thereof, therapeutically effective mutations, and humanized variants thereof. The method may include therapeutically effective formulations further comprising a pharmaceutically acceptable excipient or carrier. Preferably, the method of treatment is for a human.

The humanized variant of the first antibody may, for example, be the antibody F4 (VH and VL comprising SEQ ID NOs: 36 and 37, respectively), which is a humanized version of 16F6 (VH and VL comprising SEQ ID NOs: 5 and 6, respectively). As demonstrated in Chen et al (ACS Chem Biol. 2014 Oct. 17; 9(10):2263-73. doi: 10.1021/cb5006454. Epub 2014 Aug. 26), F4 was generated via structure-derived humanization of 16F6 using a 16F6 humanization phage library designed based on the human scaffold of the vascular endothelial growth factor-specific synthetic antibody YADS1. F4 and 16F6 have been shown to bind to the same epitope on SUDV GP and provide equivalent ability to neutralize SUDV pseudo-viruses and equivalent protection (both therapeutically and prophylactically) in mice (Chen et al., ACS Chem Biol. 2014 Oct. 17; 9(10):2263-73. doi: 10.1021/cb5006454. Epub 2014 Aug. 26)). Therefore, regardless of whether 16F6 (or its variant) or F4 (or its variant) is used as the first antibody, a similar effect should be obtained when combined with the second antibody, such as X10H2 or its variant such as its humanized variant.

Based on in vitro and rodent testing, it was determined that a formulation of two SUDV-specific mAbs: (i) one base-binding (16F6) mAb; and (ii) one glycan cap-binding (X10H2) mAb, was down-selected for assessment in a macaque model of SUDV disease. This formulation, F6-H2, provided complete protection from SUDV infection in rhesus macaques when administered at 50 mg/kg on days 4 and 6 post-infection. F6-H2 is the first SUDV-specific mAb therapy to demonstrate in vivo efficacy against SUDV in macaques. As a result, a potential treatment option for managing SUDV disease in humans is provided.

An antibody-based therapy was developed for SUDV that yielded a formulation of two mAbs capable of protecting macaques from SUDV exposure when delivered after animals are tested and found to be PCR positive. Antibody down-selection criteria focused on identifying neutralizing antibodies that targeted non-competing epitopes of SUDV GP. Cross-reactivity against other ebolaviruses was also considered, but all 8 mAbs evaluated failed to bind to GPs of EBOV and BDBV. Out of a small panel of 8 antibodies, 4 competition groups were identified and only 2 groups (5 mAbs in total) had significant neutralizing activity against SUDV. It was discovered that 16F6 and X10B1 were the most potent neutralizing and protective mAbs. Both bind at the base of the GP, similar to KZ52 and ZMapp components 2G4 and 4G7 (27, 28). The 16F6 epitope consists of residues located within both GP1 and GP2 (21) and 16F6/X10B1 potency may be attributed to its ability to either “lock” GP in a pre-fusion state and impede necessary structural rearrangements required for membrane fusion or block cathepsin cleavage of GP, as is reported for KZ52 and 2G4 (29). The other neutralizing and protective mAbs, X10H2, X10F3, and X10B6, bound an epitope located on the glycan cap, an epitope similar to that targeted by ZMapp component 13C6 (27, 28). While it was not the intention to develop a ZMapp-like formulation, the down-selection strategy described herein was similar to that used to develop ZMapp which may have skewed the selection unintentionally toward ZMapp-like mAbs. Similarities in epitope specificity between ZMapp™ and RIID F6-H2 suggested that structural features of GP vulnerability to antibody therapies may be conserved across ebolavirus species, as proposed previously (28).

Based on published studies describing EBOV-specific mAb treatment of infected macaques (12-16), SUDV-infected macaques were treated twice with 50 mg/kg of the antibody formulation. Given the rapid decrease in viremia following treatment and few clinical signs of disease observed in treated macaques, it was suspected that less aggressive dosing strategies may also be effective.

Finally, minimal cross-reactivity of ZMapp and RIID F6-H2 was due to limited GP sequence homology across ebolavirus GP sequences (28-30). Many of the conserved epitopes are protected from neutralizing antibodies owing to the large, bulky mucin-like domain and the glycan cap (29). Recent antibody development efforts focused on expanding the breadth of antibody reactivity using multiple strategies. Bispecific antibody engineering, combining EBOV and SUDV GP specific mAbs, or the ‘Trojan horse’ approach of targeting conserved intramolecular epitopes has yielded broadly active therapeutic candidates with in vivo efficacy in rodents against both viruses (23, 31). Heterologous prime-boost vaccination strategies to elicit broadly-reactive mAbs targeting conserved epitopes have also been successful (30, 32, 33). Others have identified naturally developed, broadly-reactive antibodies from human survivors of EBOV or Bundibugyo virus infections (29, 34). Given their breadth of activity, new pan-ebolavirus antibody therapies incorporating these mAbs may provide a single treatment option for managing ebolavirus outbreaks caused by any known ebolavirus species or potentially novel ebolaviruses yet to emerge. However, the utility of these pan-ebolavirus antibody therapies has yet to be fully realized as all are still in early stages of development. Moreover, the availability of multiple antibody therapeutics against ebolaviruses provides an insurance policy should one be rendered ineffective due to naturally occurring or intentionally engineered viral escape mutants, just as multiple antibiotics afford protection against anti-microbial resistance.

While the preferred embodiments of the invention are described herein, it will be recognized and understood that various modifications may be made therein, and the appended claims are intended to cover all such modifications which may fall within the spirit and scope of the invention. The following examples are intended to illustrate, but not limit, the invention.

### EXAMPLES

### Example 1. Materials and Methods

### Cells and Viruses.

Vero E6 cells (ATCC) were maintained in Eagles Minimal Essential Medium (EMEM) supplemented with 5% heat inactivated fetal bovine serum (ΔFBS) and gentamicin (50 μg/ml) at 37° C., 5% CO2, and 80% humidity. Sudan virus/H. sapiens-gp-tc/SDN/1976/Boniface-USAMRIID111808 (SUDV/Bon-USAMRIID111808; ‘SUDV-Boniface 1976’) (35) was used for all in vitro and in vivo studies requiring live virus.

### Antibody Production in Mammalian Expression System.

A total of seven chimeric anti-SUDV GP full-length heavy chain and light chain sequences were designed using the variable domain amino acid sequences of 16F6, 17F6, 19F10, 5G2, X10B1, X10F3 and X10H2. All antibody sequences were constructed utilizing murine light and heavy chain signal peptides, human IgG1 heavy constant sequences and human kappa constant sequences. All genes were codon and sequence optimized to ensure high level expression in murine cells and synthetized (GeneArt/ThermoFisher Scientific). Stable NS0 cell lines were developed at BioFactura, Inc. (Frederick, Md.) using the StableFast platform and the single cholesterol selection strategy as described by Sampey, et. al. (36). Briefly, bicistronic expression plasmids were constructed coding for both heavy and light chains of each mAb. Each chain coding sequence was flanked upstream by cytomegalovirus (CMV)-derived promoters and downstream by bovine growth hormone (BGH) polyadenylation sequences (poly-A) comprising independent heavy and light chain expression cassettes. The cholesterol selection marker 17β-hydroxysteroid dehydrogenase type 7 or Hsd17β37 was regulated by an SV40 promoter and SV40 poly-A. The serum-free medium adapted cholesterol auxotrophic NS0 host cell line (NS0-SF, ECACC, Cat No. 03061601) was transfected by electroporation by either the expression plasmids and stable cell lines were selected by withdrawal of exogenous cholesterol. Best performing pools for each mAb were scaled to shaker or spinner flasks and stirred-tank single-use bioreactors (BioBLU, Eppendorf) and operated in fed-batch mode for 7-9 days. Cultures were subsequently clarified by centrifugation and filtration and mAbs were purified by a single Protein A capture step. Briefly, clarified supernatants were loaded onto Amsphere Protein A columns (JSR Life Sciences). Columns were washed with equilibration buffer (20 mM sodium phosphate, 150 mM sodium chloride) followed by a high salt wash with 500 mM sodium chloride. The mAbs were eluted with 0.2 M glycine at pH 3.0 directly into a container with 1 M Tris-HCl pH 8.0 to neutralize the eluate. Final purified mAbs were dialyzed into 1×PBS, pH 7.4 and sterile filtered. Final purified mAbs were characterized for identity, purity and endotoxin levels.

### Antibody Production in Plant Expression System.

Anti-SUDV GP IgG1 full light chain and heavy chain sequences were designed using variable domain amino acid sequences. Signal peptide derived from Nicotiana tabacum pathogenesis-related 1a (PR1a) protein (X06930) was fused with variable heavy and light chain regions to target expression of antibodies to the apoplast. The light chain variable domains of 16F6, X10B1, X10B6, X10F3 and X10H2 were fused to a human kappa constant region to produce full length chimeric light chains, while the heavy chain variable domains were fused to a gamma constant human IgG1 region to produce chimeric IgG1 heavy chains. The light chain variable domains of 19F10 and 5G2 were fused with a mouse constant region to produce full length mouse light chains, while the heavy chain variable domains were fused with a gamma constant mouse IgG1 region to produce chimeric IgG1 heavy chains. All genes were codon optimized to ensure high level expression in Nicotiana benthamiana plants and synthetized (ThermoFisher Scientific). All genes were then cloned into the binary expression vector pGR-D35S, where expression of heavy and light chains is under control of the double 35S Cauliflower mosaic virus (CaMV) promoter and Tobacco etch virus (TEV) enhancer. Agrobacterium tumefaciens strain AGL1 was transformed with the plasmids. N. benthamiana plants were vacuum infiltrated using agrobacterium cultures and aerial tissue was harvested after four to six days.

Antibodies used in the mouse studies were purified with Protein A. Biomass containing Sudan specific mAbs were homogenized after harvest in a Tris based buffer followed by detergent extraction for 20 minutes. The extract was clarified by centrifugation and filtration prior to loading onto on a MabSelect prepacked column (GE Healthcare). Elution was achieved with a citrate buffer, pH 3.0 directly into a container with 2 M Tris pH 9.0 to neutralize the eluate. The recovered mAbs were dialyzed overnight into 1×PBS, pH 7.4, concentrated in a centrifugal device to >than 1 mg/mL and terminally filtered with a 0.22 μm PVDF membrane prior to storage at ≤−60° C. Plant expressed mAbs for use in the NHP studies were captured on Protein A and held overnight in the neutralized eluate. The following day, the eluate was diluted to a pH of approximately 5.6 and loaded onto an SP column and eluted with 150 mM salt. The SP eluate was concentrated and buffer exchanged with ultrafiltration and diafiltration prior to loading on to Capto-Q and collected in the unbound fraction. The final mAb was augmented with polysorbate-80 to 0.005%, aseptically filtered (0.22 μm), aliquoted and held frozen at ≤−60° C. All chromatography media was obtained from GE Healthcare. Final, purified mAbs for NHP studies were released based on attributes of identity, purity (≥90%) and endotoxin ≤0.1 EU/mg among other attributes.

### Enzyme Linked Immunosorbent Assay (ELISA).

SUDV or SUDV GP-specific IgG titers were determined by ELISA using irradiated SUDV or recombinant SUDV GP (National Cancer Institute, Protein Expression Lab) as previously described (12). Briefly, polyvinyl chloride ELISA plates (Dynatech Laboratories) were coated with irradiated virus or recombinant GP, diluted in PBS, overnight at 4° C. before blocking with 5% milk protein in PBS/0.02% Tween 20 at room temperature. Monoclonal antibodies or serum samples were serially diluted in blocking buffer and added to antigen coated plates for two hours at room temperature. Plates were washed with wash buffer (PBS/0.02% Tween 20) before adding horseradish peroxidase conjugated goat anti-human IgG (Rockland) for 1 hour at room temperature. Following a final wash, 2, 2′-Azinobis [3-ethylbenzothiazoline-6-sulfonic acid]-diammonium salt (ABTS) substrate (Kirkegaard and Perry Laboratories, Inc.) was added and absorbance values were read at 405 nm using a Spectramax® plate reader after 30 minutes (Molecular Devices, LLC). For serum sample antibody end titers, cut-off values for each dilution were defined using pre-challenge serum samples from each individual macaque. Cut-off values for each dilution were calculated using absorbance values of pre-challenge serum for that dilution and the following formula: average pre-vaccination serum absorbance+3× standard deviation. End-point titers for each serum sample are expressed as the last dilution to exceed the cut-off value for a given dilution.

### Competitive ELISA.

Polyvinyl chloride ELISA plates (Dynatech Laboratories) were coated with SUDV GP or irradiated SUDV (for 19F10 and 5G2), diluted in PBS, overnight at 4° C. before blocking with 5% milk protein in PBS/0.02% Tween 20 at room temperature. Human IgG versions of indicated mAbs were diluted to 0.1 mg/ml in PBS and added to antigen coated plates for 20 minutes at room temperature. Murine IgG versions of indicated mAbs were diluted to 0.1 mg/ml in PBS and added to wells containing human IgGs or PBS alone for 20 minutes at room temperature. Plates were washed with wash buffer (PBS/0.02% Tween 20) before adding horseradish peroxidase conjugated goat anti-mouse IgG (Rockland, Glibertsville, Pa.) for 1 hour at room temperature. Following a final wash, 2, 2′-Azinobis [3-ethylbenzothiazoline-6-sulfonic acid]-diammonium salt (ABTS) substrate (Kirkegaard and Perry Laboratories, Inc.) was added and absorbance values were read at 405 nm using a Spectramax® plate reader after 30 minutes (Molecular Devices, LLC).

### SPOTS Membrane Assay.

SPOTS membrane assays were completed using Genosys SPOTs (Sigma-Aldrich) membranes in accordance with the manufacturer's instructions. Briefly, SPOTs membranes, coated with 13-mer overlapping linear peptides (95% purity) from SUDV/Boniface and SUDV/GuIu, and were incubated sequentially with SUDV-specific mAbs and β-galactosidase conjugated anti-murine IgG secondary antibody. Signal development solution was added to the membrane before washing with PBS and imaging with a ChemiDoc™ XRS+ imaging system (Bio-Rad Laboratories, Inc.).

### Microneutralization Assay.

Antibodies were diluted to indicated concentrations in culture media and incubated with SUDV for 1 h. VeroE6 cells were exposed to antibody/virus inoculum at an MOI of 0.5 plaque-forming unit (PFU)/cell for 1 h. Antibody/virus inoculum was then removed and fresh culture media was added. At 48 h post-infection, cells were fixed with formalin and blocked with 1% bovine serum albumin. SUDV infected cells and uninfected controls were incubated with SUDV GP-specific mAb 3C10 (USAMRIID). Cells were washed with PBS prior to incubation with goat anti-mouse IgG conjugated to Alexa 488. Cells were counterstained with Hoechst stain (Invitrogen), washed with PBS and stored at 4° C. Infected cells were quantitated by fluorescence microscopy and automated image analysis. Images were

### Mouse Studies.

4-8-week-old male and female type I IFN α/β receptor knockout mice (IFNAR−/−) (Jackson Labs) were exposed to a target dose of 1000 pfu (actual dose: 234-910 pfu) of SUDV via intraperitoneal (I.P.) injections. Mice were treated I.P. on days 1 and 4 post-exposure with PBS vehicle or 200 mg of antibody diluted in 0.2 ml of PBS. Animals were observed daily for clinical signs of disease and lethality. Daily observations were increased to a minimum of twice daily while mice were exhibiting signs of disease. Moribund mice were humanely euthanized on the basis of IACUC approved criteria.

### Rhesus Macaque Study.

Male and female rhesus macaques (4-7.8 kg) were randomly assigned to treatment groups, with even distribution of male and female macaques, and study personnel remained blinded to all treatment groups. Macaques were inoculated intramuscularly (I.M.) with a target dose of 1000 pfu (actual dose: 1750 pfu) of SUDV. Experimental macaques (n=4) were treated intravenously (I.V.) with a formulation of plant produced 16F6 and X10H2 at 50 mg/kg total (25 mg/kg per antibody) on days 4 and 6 post-exposure. Control macaques 1 and 2 received an equal volume to weight ratio of vehicle I.V. on days 4 and 6 post-exposure. Control macaques 3 and 4, for a separate treatment cohort run concurrently, received an equal volume to weight ratio of vehicle I.V. on day 5 post-exposure. Blood samples were collected from experimental macaques and control macaques 1 and 2 on days 0, 4, 6, 8, 11, 14, 21, and 28 post-exposure and from control macaques 3 and 4 on days 0, 3, 5, 8, 11, 14, 21, and 28 post-exposure. Blood samples were used to evaluate viremia, blood chemistries, and hematology. Physical exams were also completed on blood collection days to assess the overall physical condition of the macaque and disease progression. Cage side observations were completed at least once daily through day 28 to assess the general disposition of each animal and disease progression. Clinical scores for each macaque were derived from cage side observations of awake animals and physical observations completed on anesthetized animals. Animals were assessed and given a cumulative score based on numerous behavioral and physical parameters, including responsiveness, petechial rash, temperature, weight change, gastrointestinal and renal output, anorexia, respiration, dehydration, and edema. Higher clinical scores indicate more severe signs of disease. Macaques determined to be moribund, in accordance with IACUC approved criteria, were promptly euthanized.

### Hematology and Blood Chemistry Analysis.

Phlebotomy was performed while the animals were anesthetized, and blood was collected from the femoral vein using a venous blood collection system (Becton Dickinson). Hematological values for blood samples collected in tubes containing EDTA were determined using a Coulter® Ac-T diff™ analyzer (Beckman Coulter). Serum chemistry was analyzed using Piccolo® General Chemistry 13 reagent discs and a Piccolo Xpress® point-of-care blood analyzer (Abaxis).

### Animal Welfare Statement.

Murine and macaque challenge studies were conducted under IACUC-approved protocols in compliance with the Animal Welfare Act, PHS Policy, and other applicable federal statutes and regulations relating to animals and experiments involving animals. The facility where these studies was conducted (USAMRIID) is accredited by the Association for Assessment and Accreditation of Laboratory Animal Care, International (AAALAC) and adhere to principles stated in the Guide for the Care and Use of Laboratory Animals, National Research Council, 2011.

### Statistical Analysis.

Antibody binding curves were evaluated using a four-parameter nonlinear regression analysis under assumption of normality. Dose-response neutralization curves were evaluated using nonlinear regression analysis (normalized response-variable slope) to determine IC50 concentrations under assumption of normality. Analysis of mouse weight change was completed by Two-way ANOVA and Dunnett's multiple comparisons test at each time point relative to control treated animals. Significance reported were lowest p values for any time point for given antibody treatment group. Survival curves were evaluated using Fisher's exact test. Alpha was 0.05 for all statistical tests. All analyses were carried out using Graph Pad Prism.

### Example 2. Chimerization and Production of SUDV-Specific mAbs

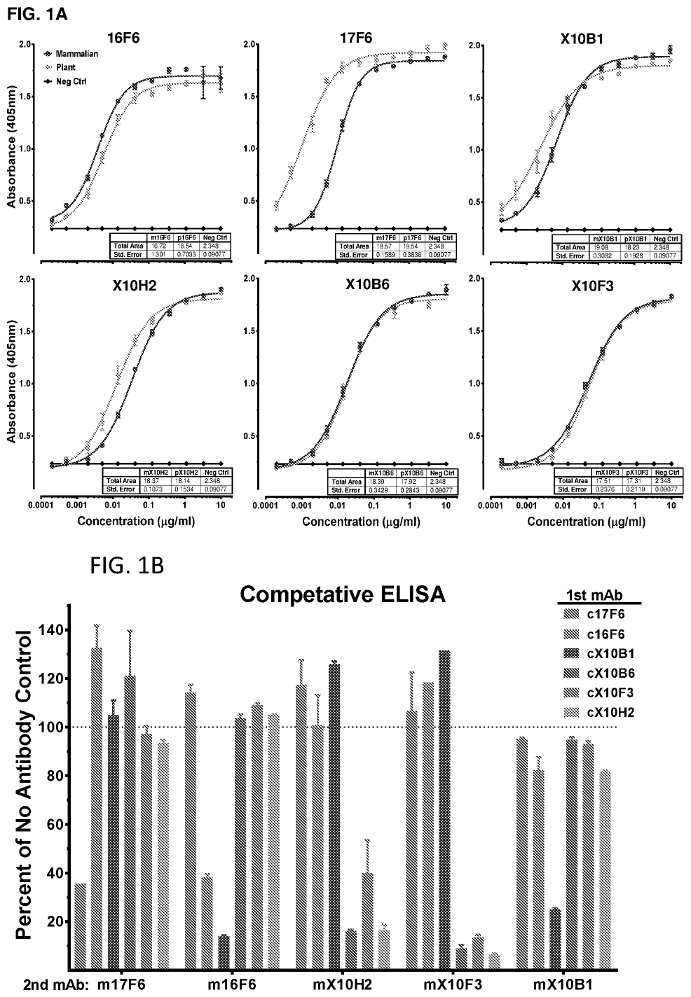

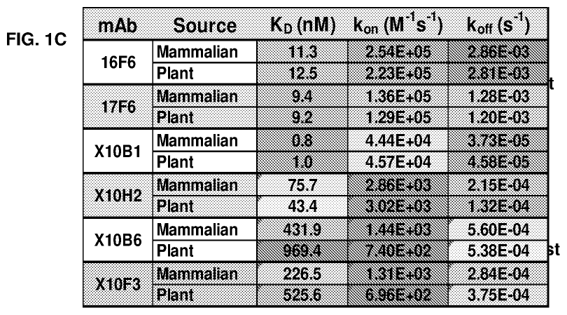

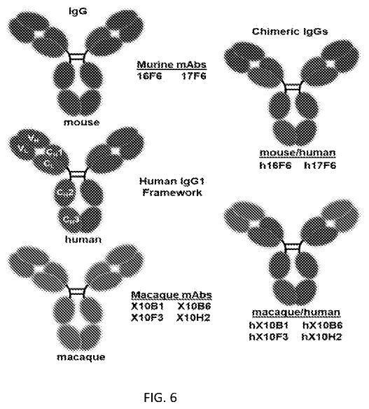

To develop SUDV-specific mAbs with the potential for human use two existing libraries of antibodies specific for SUDV GP were leveraged. The first library consisted of monoclonal antibodies derived from BALB/c mice immunized with Venezuelan equine encephalitis virus replicon particle (VRP) vaccine expressing SUDV/Boniface GP, as described previously (21). The second library was comprised of novel single chain variable fragment (scFv) antibodies isolated from macaques immunized with VRP vaccine expressing SUDV/Boniface GP (22). Also, human IgG1 chimeric mAbs were generated by cloning variable heavy (VH) and variable light (VL) chains from the murine and macaque antibodies into human IgG1 constant heavy and constant light chain expression vectors (FIG. 6, Table 1). Nucleotide sequences encoding each chimeric mAb were codon optimized for expression in either tobacco (Nicotiana) plants or mammalian NS0 cells. Human IgG1 chimeric heavy and light chains were co-expressed in either plants or mammalian cells, and IgGs were purified using Protein A affinity resin. The binding specificity of each humanized mAb was evaluated using recombinant SUDV GP or irradiated SUDV coated ELISA plates (FIG. 1A).

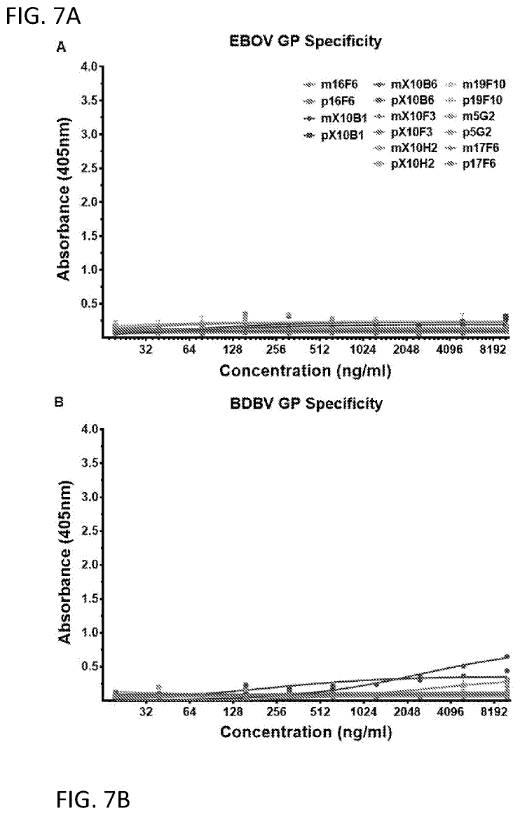

Following chimerization, SUDV-specific mAbs retained similar binding profiles relative to their parent antibodies (data not shown). Overall, binding profiles of plant and mammalian derived mAbs were equivalent with the exception of 17F6, 19F10, and X10H2. Plant 17F6 bound more efficiently to SUDV GP compared to mammalian 17F6, yet the opposite was true against irradiated SUDV. For both 19F10 and X10H2, plant derived mAbs bound irradiated SUDV (19F10) or SUDV GP (X10H2) more efficiently than mammalian derived product. Interestingly, 5G2 and 19F10 specificity was limited to irradiated SUDV, and binding of 16F6 to recombinant GP was significantly weaker than other SUDV GP-specific mAbs despite being a known potent neutralizer of SUDV (21). Fast protein liquid chromatography (FPLC) of the recombinant SUDV GP used in our assays determined that the protein is primarily monomeric, unlike the trimeric spike present on virion particles (data not shown). The 16F6 epitope bridges two monomers of the trimeric spike (21), a quaternary epitope not maintained in the monomeric SUDV GP, leading to abrogation of 16F6 binding. While the epitopes for 19F10 and 5G2 are unknown, reduced affinity to recombinant SUDV GP suggests that their epitopes may too be quaternary in nature. Cross-reactivity of each mAb against recombinant EBOV GP or Bundibugyo virus (BDBV) GP was evaluated and none of the mAbs were reactive against either ebolavirus species (FIG. 7).

### Example 3. In Vitro Characterization of SUDV-Specific mAbs

Next, the competition groups were defined for each of the eight SUDV-specific mAbs using competitive ELISAs. Chimeric mAbs were incubated with SUDV GP coated plates (or irradiated SUDV for 5G2 and 19F10) prior to the addition of the murine version of each mAb. Binding of murine mAb alone was compared to observed binding levels in the presence of the chimeric mAbs to identify any competitors. 17F6, 19F10, and 5G2 did not compete with any other mAbs in the panel (FIG. 1B). However, X10H2, X10F3, and X10B6 appear to belong to the same competition group (FIG. 1B). X10B1 blocked 16F6 binding, but 16F6 did not significantly block X10B1 binding, indicating a directional competition (FIG. 1B). One possible explanation for this discrepancy is epitope overlap or steric hindrance related to close proximity between X10B1 and 16F6 epitopes. Instead, X10B1 may have a lower KD for the epitope than 16F6 and would therefore outcompete 16F6 for epitope binding. Alternatively, the binding angle of X10B1 may preclude 16F6 from binding yet the X10B1 epitope remains accessible following 16F6 binding. Finally, X10B1 binding may induce conformational changes to GP that alter the 16F6 epitope. 16F6 binding to GP first, may stabilize the epitope while still allowing X10B1 to bind.

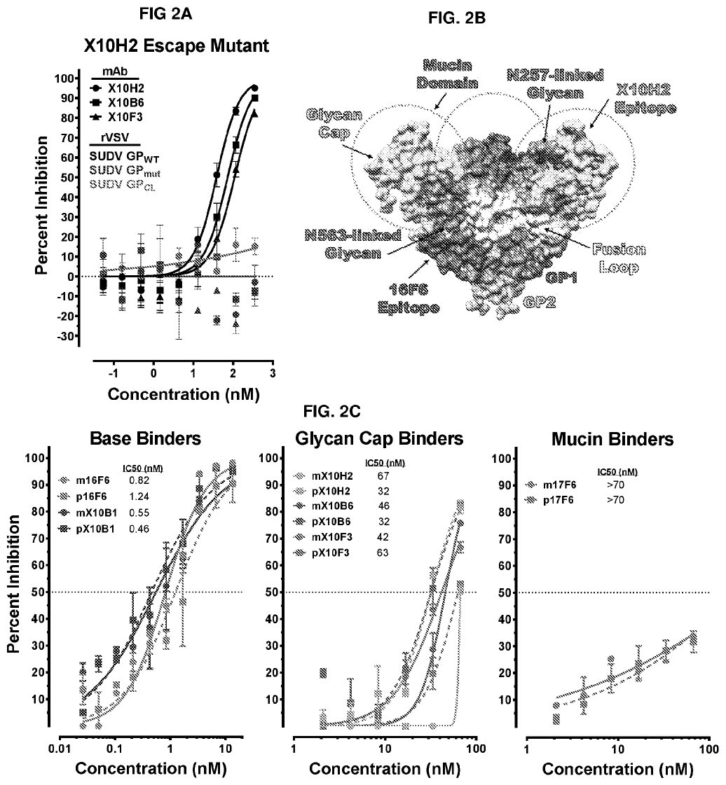

Neutralizing activity of each mAb against authentic SUDV/Boniface was evaluated using a microneutralization assay (23). Base binding antibodies 16F6 and X10B1 were the most potent neutralizers of SUDV with sub-nanomolar (nM) half-maximal inhibitory concentrations (IC50) (FIG. 2C). Competing antibodies X10H2, X10F3, and X10B6 were all weak neutralizers with comparable IC50 values between 32 and 67 nM (FIG. 2C). Non-competing antibodies 17F6, 19F10, and 5G2 failed to neutralize SUDV even at the highest concentration tested (FIG. 2C). No significant difference in neutralizing activity was observed between plant and mammalian derived mAbs.

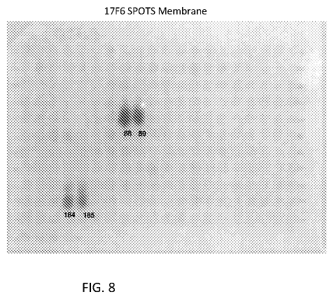

Mapping binding footprints for each SUDV-specific mAb was attempted. The binding epitope for 16F6 was previously determined by x-ray crystallography to be located at the base of the GP trimer, overlapping GP1 and GP2 (21). Given their competition by ELISA, both 16F6 and X10B1 were assigned as GP base binders. SPOTS membranes coated with 13 amino acid-long (13mer) linear peptides of SUDV GP from Boniface and GuIu isolates were employed to identify mAbs with linear epitope specificity. 17F6 was the only mAb that recognized a linear epitope, a mucin-like domain (MLD) amino acid sequence 353LH (V/I) PEGETT361 (SEQ ID NO.:44) (FIG. 8). To map the biding site of X10H2 and competing antibodies, Viral escape variants from antibody neutralization were selected by serially passaging recombinant vesicular stomatitis virus expressing SUDV/Boniface GP (rVSV-SUDV) in the presence of X10H2 until resistance to neutralization was observed. Following passage four, the viral population was plaque purified and sequenced to identify the mutations in SUDV GP that engendered viral neutralization escape. A single such viral mutant with a substitution of glutamine (Q) with lysine (K) at position 264 in the glycan cap of GP (FIG. 2A) was identified. Consistent with other glycan cap binding antibodies, X10H2 was unable to neutralize cleaved SUDV GP further corroborating glycan cap binding specificity of X10H2 and competing mAbs (FIG. 2A). Lack of neutralizing activity for 19F10 and 5G2 precluded us from generating similar escape mutants, and their binding specificities remain undefined. The SUDV GP structure with defined structural elements is depicted in FIG. 2B. Amino acid residues of the 16F6 epitope, located near the base of the GP trimer, are colored purple and are presumed to overlap or be in close proximity to residues of the X10B1 epitope. The residue at position 264, critical for X10H2 neutralization, is colored red with a 10A zone around the residue colored yellow to approximate the outline of the mAb's putative epitope. The binding residues for X10H2 and the two competing mAbs, X10F3 and X10B6, are likely to be located within the 10A zone.

### Example 4. Protective Efficacy of SUDV-Specific mAbs in Mice

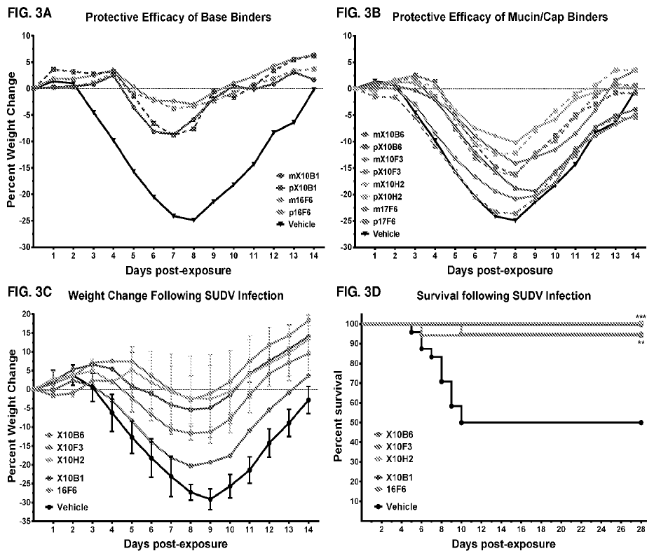

To evaluate the protective efficacy of SUDV-specific mAbs, the interferon-alpha/beta receptor knockout (IFNAR−/−) mouse model of SUDV disease (24) was utilized. The efficacy of plant and mammalian derived mAbs were compared using 5-8-week-old IFNAR−/− mice, for which the primary metric of protection is reduced weight loss following SUDV challenge. Age matched IFNAR−/− mice were treated intraperitoneally (I.P.) with plant or mammalian derived mAbs on days 1 and 4 post-exposure to 1000 plaque forming units (pfu) of SUDV via I.P. inoculation, and weight loss was monitored. Base binding mAbs 16F6 and X10B1 reduced weight loss significantly relative to vehicle control animals with 16F6 performing slightly better than X10B1 (FIG. 3A). Mucin domain binding mAb 17F6 afforded no protective benefit against SUDV challenge and was dropped from further consideration (FIG. 3B). Of the glycan cap binding mAbs, X10H2 was the most protective, followed by X10F3, then X10B6 (FIG. 3B). In all cases, no significant difference in weight loss was observed between mice treated with plant or mammalian derived mAbs.

Next, down-selecting lead mAb candidates from the base binding and glycan cap binding groups was conducted. For these studies, 4-week-old IFNAR−/− mice were used, which is a lethal model of disease. As before, IFNAR−/− mice were treated with mAbs on days 1 and 4 post-exposure to 1000 pfu of SUDV, and weight loss and survival were monitored. In this model, base binders 16F6 and X10B1 significantly reduced weight loss relative to vehicle control treated mice (p<0.01) (FIG. 3C) and provided 90-100% protection (p<0.01) (FIG. 3D). 16F6 was selected for further development given its slightly better and more consistent reduction in weight loss. Glycan cap binding mAbs X10H2, X10F3, and X10B6 also provided 90-100% protection (p<0.01) (FIG. 3D), but only X10H2 limited weight loss significantly (p<0.05) (FIG. 3C), and was therefore selected for further development.

### Example 5. Protective Efficacy of SUDV-Specific Formulation in Rhesus Macaques

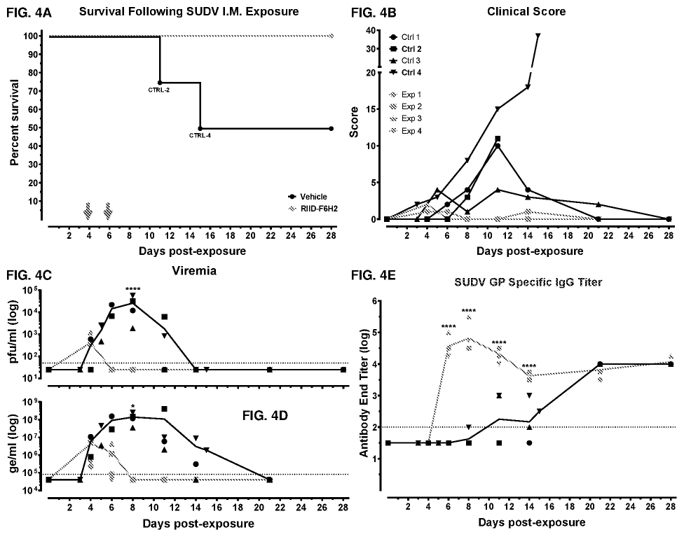

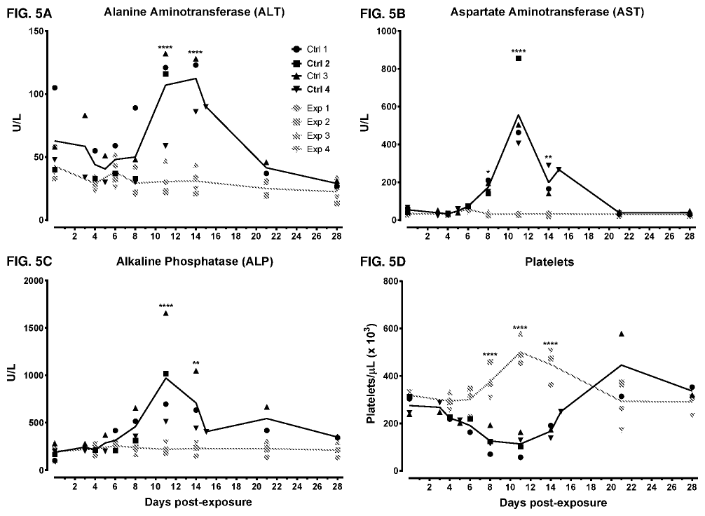

The protective efficacy of the SUDV-specific antibody formulation comprised of 16F6 and X10H2, hereafter F6-H2, was evaluated in nonhuman primates using the rhesus macaque model of Sudan virus disease (25, 26). Adult rhesus macaques were equally randomized into treatment groups and blinded to study personnel for the duration of the study. Macaques were exposed to a target dose of 1000 pfu of SUDV/Boniface via intramuscular injection on day 0 of the study. On days 4 and 6 post-exposure, 4 experimental macaques were treated intravenously (I.V.) with plant-derived F6-H2 at 25 mg/kg per mAb (50 mg/kg total). Plant-produced mAbs were used in this study due to availability of product at the required quantity and the previously established equivalent protective efficacy (FIG. 2C). Control macaques 1 and 2 were treated I.V. with an equivalent volume to weight ratio of diluent on days 4 and 6. Control macaques 3 and 4 from a concurrent study were treated with an equivalent volume to weight ratio of diluent on day 5 only. Macaques were observed daily to assess overall health, behavior, and diet. In addition, physical examinations and blood collections were completed approximately every 3-4 days to assess clinical disease progression. All F6-H2 treated macaques survived SUDV exposure with little to no clinical signs of disease. By contrast, 50% of control macaques succumbed to SUDV infection and all displayed severe clinical signs of disease associated with ebolavirus infection (FIG. 4A-FIG. 4B). All control macaques were PCR positive for SUDV by day 4 post-exposure (FIG. 4C); by day 6, all control macaques had detectable viremia by plaque assay (FIG. 4D). Viral titers in control macaques peaked between days 6 and 8 at 4 to 5 log 10/ml in 3 of 4 macaques. Peak viral titer for Ctrl 3 was approximately 1 log 10 lower at just over 3 log 10/ml, which may explain less severe disease symptoms observed in this macaque. All experimental macaques were PCR positive for SUDV prior to receiving the first antibody treatment on day 4 post-exposure (FIG. 4C), and viremia was detected by plaque assay in 2 of 4 experimental macaques on day 4 (FIG. 4D). Following F6-H2 treatment on day 4, viral titers decreased rapidly in all 4 experimental macaques and remained below the limit of plaque assay detection through the end of the study. All 4 experimental macaques were PCR negative by day 8 post-exposure, two days after the last antibody treatment. As anticipated, SUDV GP-specific IgG titers increased rapidly following antibody delivery and remained elevated through day 28 (FIG. 4E). A de novo SUDV GP-specific IgG response was detectable in control macaques as early as day 8 post-exposure; by day 21 post-exposure, surviving control macaques had similar titers relative to treated macaques (FIG. 4E). All control macaques had elevated serum levels of ALT, AST, ALP (FIG. 5A-FIG. 5C), and thrombocytopenia (FIG. 5D) during the acute phase of disease.

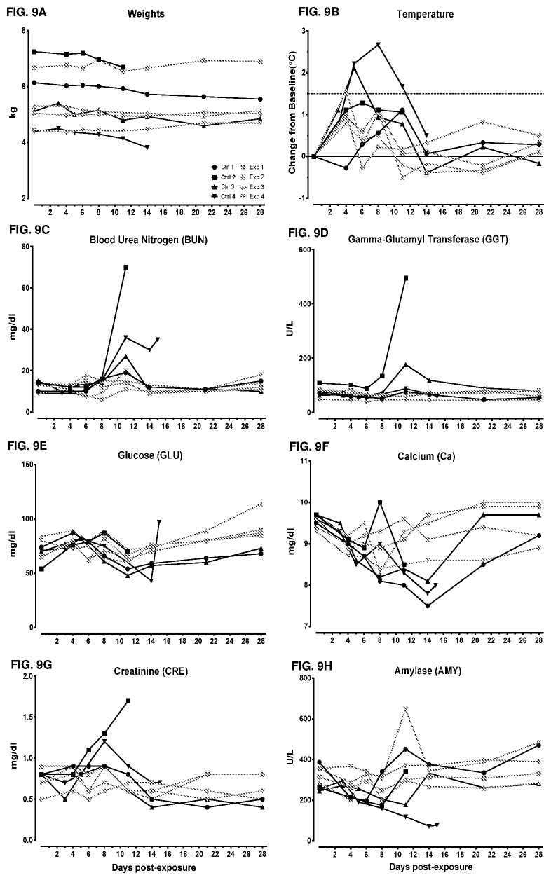

Elevated levels of BUN, GGT, and CRE, and reduced levels of AMY were also observed in serum of control animals that succumbed (FIG. 9). Experimental macaques treated with F6-H2 were free of any abnormal serum chemistries or hematology associated with ebolavirus disease (FIG. 5, FIG. 9).

The protective efficacy will also be tested using the same protocols using a composition comprising F4 and X10H2. Since F4 is a humanized version of 16F6 and binds to the same epitope as and provides equivalent in vitro neutralization and in vivo efficacy in mice to that of 16F6 (Chen et al., ACS Chem Biol. 2014 Oct. 17; 9(10):2263-73. doi: 10.1021/cb5006454. Epub 2014 Aug. 26)), the composition of F4 and X10H2 will provide an equivalent effect as the composition of 16F6 and X10H2.

TABLE 1
SEQ ID NO: 5 (16F6 Heavy Chain)
EVQLVESGGGLVTPGGSLKLSCAASGFAFNYYDMFWVRQNTEKRLEW
VAYINSGGGNTYYPDTVKGRFTISRDNAKKTLFLQMSSLRSEDTAMY
YCARQLYGNSFFDYWGQGTSLTV
SEQ ID NO: 6 (16F6 Light Chain)
DIVMTQSHKFMSTSVGDRVTITCKASQDVTTAVAWYQQKPGHSPKLL
IYWASTRHTGVPDRFTGSGSGTDFTLTLNSVQAEDLALYYCQQHYST
PLTFGAGTKLEL
SEQ ID NO: 7 (19F10 Heavy Chain)
MEWSWVFLFLLSGTAGVLSEVQLQQSGPELVKPGASVKISCKASGYS
FTGYYMHWVKQSHVKSLEWIGRINPYNGATSYNQNFKDKASLTVDKS
SSTAYMELHSLTSEDSAVYYCARPDYTSSFAYWGQGTLVTVSAAKTT
PPPVYPXXPGSLG
SEQ ID NO: 8 (19F10 Light Chain)
MVSSAQFLGLLLLCFQGTRCDIQMTQTTSSLSASLGDRVTISCRASQ
DISNYLNWYQQKPDGTVKLLIYYTSRLHSGVPSRFSGSGSGTDYSLT
ISNLEQEDIATYFCQQGNTLPWTFGGGTKLEIKRADAAPTVSIFSPS
SKL
SEQ ID NO: 9 (5G2 Heavy Chain)
MEWXWVILFLMAVVTGVNSEVQLQQSGAELVKPGASVKLSCTASGFN
IKDTYIHWVKQRPEEGLEWIGRIDPANDNIKFDPKFQGKATITADTS
SNTAYLQLLSLTSEDTAVYFCARTNGLDYWGQTTLTVSSAKTTPPSV
YPLXPGKAW
SEQ ID NO: 10 (5G2 Light Chain)
MKLPVRLLVLMFWIPASSSDVVMTQTPLSLPVSLGDQASISCRSSQS
LLHSNGNTYSHWYLQKPGQSPKLLIYKVSNRLSGVPDRFSGSGSGTD
FTLKISRVEAEDLGVYFCSQSTHVPYTFGGGTKLEIKRADAAPTVSI
FPPSSKLG
SEQ ID NO: 11 (17F6 Heavy Chain)
MMVLSLLYLLTVVPGILSDVQLQESGPGLVKPSQTVSLTCTVTGISI
TAGNYRWSWIRQFPGNKLEWIGYIYYSGTIAYNPSLTSRTAITRDSS
KNQFFLEMNSLTAEDTATYYCARDRGWLLLDYWGQGTTLTVSSAKTT
APSVYPLAPGSL
SEQ ID NO: 12 (17F6 Light Chain)
MKLPVRLLVLMFWIPASSSDVLMTQTPLSLPVSLGDQASISCRSSQT
IVHSNGNTYLEWYLQKPGQSPKLLIYKVSSRFSGVPDRFSGSGSGTD
FTLKISRVEAEDLGVYYCFQGSHFPYTFGGGTKLEIKRADAAPTVSI
FPPSSKL
SEQ ID NO: 13 (X10H2 Heavy Chain)
QVQLEQSGAEVKKPGTSVKVSCKASGFTFGSDAISWVRQAPGQGLEW
MGVIIPVVGARTYAEKFQGRITITADTSTSTVFMDLSSIRSDDTAVY
YCAREGARAATGHYKSMDIWGRGTLV
SEQ ID NO: 14 (X10H2 Light Chain)
EIELTQSPSSLYASVGDKVTITCRASQGISTYLNWYQQKPGKAPKLL
IYAASSLQSGVPSRFSGSGSGTEFTLTISSLQPEDFAAVYCQQYNSY
PRTFGQGTKVEIK
SEQ ID NO: 15 (X10B1 Heavy Chain)
EVQLLESGPSLVKPSETLTLTCAVSGASISGDYLNWIRQPPGKGLEW
IGGIFGSGGSTDYSPSFKSRVTISTDTSKNQFSLKLTSMTTADTAVY
FCATALRGARFDAFDFWGQGRRV
SEQ ID NO: 16 (X10B1 Light Chain)
DIELTQSPATLSLSPGERATLSCRASQSVSTYLAWYQKKPGQAPRLL
FYGASNRATGIPDRFSGRRSGTDFTLTISSLEPEDVGVYYCQQYNKW
NSFGQGTKVEIK
SEQ ID NO: 17 (X10F3 Heavy Chain)
QVQLEQSGAEVKKPGTSVKVSCKASGFFTFGSDAISWVRQAPGQGLE
WMGVIIPVVGARTYAEKFQGRITITADTSTSTVFMDLSSLRSDDTAV
YYCAREGARAATGHYKSMDIWGRGRTLV
SEQ ID NO: 18 (X10F3 Light Chain)
ELQMTQSPSSLSASVGDRVTITCRASQDIGPFLNWYQHKPGKPPKLL
IYDVSNLQDGVPSTFSGSGSGTDFTLTISNLQPEDFATYYCLHYANY
PRTFGPGTKVEIK
SEQ ID NO: 19 (X10B6 Heavy Chain)
QVQLEQSGAEVKKPGTSVKVSCKASGFFTFGSDAISWVRQAPGQGLE
WMGVIIPVVGARTYAEKFQGRITITADTSTSTVFMDLSSLRSDDTAV
YYCAREGARAATGHYKSMDIWGRGRTLV
SEQ ID NO: 20 (X10B6 Light Chain)
ELLMTQSPSSLSASVGDRVTITCRASQGISTYLSWFQQKPGKPPKLL
IYLTSSLEDGVPSRFSGAGSGTEFTLTISNLQPEDFASYYCLQYQSY
PRTFGPGTKVEIK
SEQ ID NO: 21 (hIgG1 CH)
ASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKV
DKRVEPKSCDKTHTCPPCPAPELLGGPSVFLFPPKPKDTLMISRTPE
VTCVVVDVSHEDPEVKFNWYVDGVEVHNAKTKPREEQYNSTYRVVSV
LTVLHQDWLNGKEYKCKVSNKALPAPIEKTISKAKGQPREPQVYTLP
PSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG
K
SEQ ID NO: 22 (hIgG1 CL)
RTVAAPSVFIFPPSDEQLKSGTASVVCLLNNFYPREAKVQWKVDNAL
QSGNSQESVTEQDSKDSTYSLSSTLTLSKADYEKHKVYACEVTHQGL
SSPVTKSFNRGEC

X10H2 sequences:
Light Chain variable region (VL) -
(SEQ ID NO. 14) 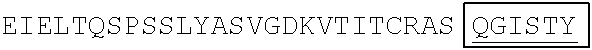
LNWYQQKP
GKAPKLLIY 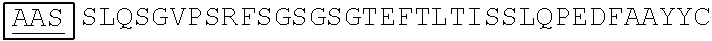
AAYYC 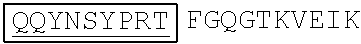
Heavy Chain variable region (VH) -
(SEQ ID NO. 13) 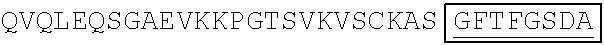
ISWVR
QAPGQGLEWMGV 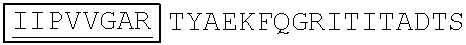
TSTVFMDLSSLRSDDTAVYYCAREG 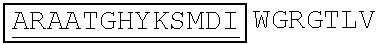
Whole scFv Sequence for X10H2:
(SEQ ID NO. 23)

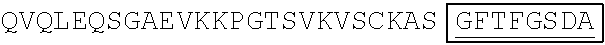

ISWVRQAPGQGLEWMGV

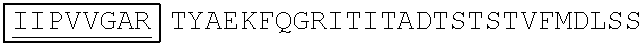

LRSDDTAVYYCAREG 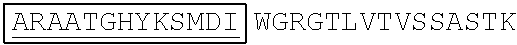
GPKLEEGEFSEARVEIELTQSP 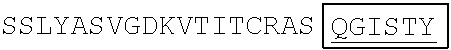
LNWYQQKPGKAPKLLIYSLQSGVPSRFSGSGS

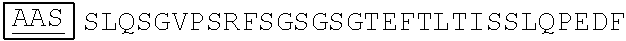

AAYYC 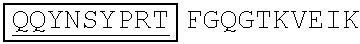
Bold & italicized_ - Linker sequence
(only used in scFv manufacture/testing) [symbol]
- CDR locations
(read as CDR 1, 2, and 3 from left to right as you read the sequence)
X10H2 CDRs
(SEQID NO: 24)
QGISTY
(SEQ ID NO: 25)
AAS
(SEQ ID NO: 26)
QQYNSYPRT
(SEQ ID NO: 27)
GFTFGSDA
(SEQ ID NO: 28)
IIPVVGAR
(SEQ ID NO: 29)
ARAATGHYKSMDI
16F6 sequences
VH sequence:
(SEQ ID NO: 5)
EVQLVESGGGLVTPGGSLKLSCAASGFAFNYYDMFWVRQNTE
KRLEWVAYINSGGGNTYYPDTVKGRFTISRDNAKKTLFLQMSS
LRSEDTAMYYCARQLYGNSFFDYWGQGTSLTV
VL sequence:
(SEQ ID NO: 6)
DIVMTQSHKFMSTSVGDRVTITCKASQDVTTAVAWYQQKPGH
SPKLLIYWASTRHTGVPDRFTGSGSGTDFTLTLNSVQAEDLALY
YCQQHYSTPLTFGAGTKLEL_
Bold = CDR locations
Possible Linker sequence:
TVSAAKTTPPSXYXLXPGSL
16F6 CDRS
VH CDR1 sequence:
(SEQ ID NO: 30)
GFAFNYYDMF
VH CDR2 sequence:
(SEQ ID NO: 31)
YINSGGGNTYYPDTV
VH CDR3 sequence:
(SEQ ID NO: 32)
QLYGNSFFDY
VL CDR1 sequence:
(SEQ ID NO: 33)
DVTTA
VL CDR2 sequence:
(SEQ ID NO: 34)
WASTR
VL CDR3 sequence:
(SEQ ID NO: 35)
CQQHYSTPLT
F4 sequences
VH sequence:
(SEO ID NO: 36)
EVQLVESGGGLVQPGGSLRLSCAASGFAFNYYDMFWVRQAPG
KGLEWVAYIKPGGGNTYYADSVKGRFTISADTSKNTAYLQMN
SLRAEDTAVYYCARQLYGNSFFDYWGQGTLVTVSS
VL sequence:
(SEQ ID NO: 37)
DIQMTQSPSSLSASVGDRVTITCKASQDVTTAVAWYQQKPGKA
PKLLIYWASTRHTGVPSRFSGSGSGTDFTLTISSLQPEDFATYYC
QQHYSTPLTFGQGTKVEIK_
Bold = CDR location
F4 CDRs
VH CDR1 sequence:
(SEQ ID NO: 38)
GFAFNYYDMF
VH CDR2 sequence:
(SEQ ID NO: 39)
YIKPGGGNTYYADSV
VH CDR3 sequence:
(SEQ ID NO: 40)
QLYGNSFFDY
VL CDR1 sequence:
(SEQ ID NO: 41)
DVTTA
VL CDR2 sequence:
(SEQ ID NO: 42)
WASTR
VL CDR3 sequence:
(SEQ ID NO: 43)
CQQHYSTPLT

### REFERENCES

1. J. H. Kuhn et al., Virus nomenclature below the species level: a standardized nomenclature for natural variants of viruses assigned to the family Filoviridae. Arch Virol 158, 301-311 (2013).
2. CDC.
3. CDC.
4. S. T. Agnandji et al., Phase 1 Trials of rVSV Ebola Vaccine in Africa and Europe. The New England journal of medicine 374, 1647-1660 (2016).
5. P. I. W. Group et al., A Randomized, Controlled Trial of ZMapp for Ebola Virus Infection. The New England journal of medicine 375, 1448-1456 (2016).
6. A. Huttner et al., The effect of dose on the safety and immunogenicity of the VSV Ebola candidate vaccine: a randomised double-blind, placebo-controlled phase ½ trial. Lancet Infect Dis 15, 1156-1166 (2015).
7. J. E. Ledgerwood et al., Chimpanzee Adenovirus Vector Ebola Vaccine. The New England journal of medicine 376, 928-938 (2017).
8. J. A. Regules et al., A Recombinant Vesicular Stomatitis Virus Ebola Vaccine. The New England journal of medicine 376, 330-341 (2017).
9. F. C. Zhu et al., Safety and immunogenicity of a recombinant adenovirus type-5 vector-based Ebola vaccine in healthy adults in Sierra Leone: a single-centre, randomised, double-blind, placebo-controlled, phase 2 trial. Lancet 389, 621-628 (2017).
10. W. B. Oswald et al., Neutralizing antibody fails to impact the course of Ebola virus infection in monkeys. PLoS Pathog 3, e9 (2007).
11. P. B. Jahrling, J. B. Geisbert, J. R. Swearengen, T. Larsen, T. W. Geisbert, Ebola hemorrhagic fever: evaluation of passive immunotherapy in nonhuman primates. J Infect Dis 196 Suppl 2, S400-403 (2007).
12. J. M. Dye et al., Post exposure antibody prophylaxis protects nonhuman primates from filovirus disease. Proc Natl Acad Sci USA 109, 5034-5039 (2012).
13. G. G. Olinger, Jr. et al., Delayed treatment of Ebola virus infection with plant-derived monoclonal antibodies provides protection in rhesus macaques. Proc Natl Acad Sci USA 109, 18030-18035 (2012).
14. X. Qiu et al., Successful treatment of ebola virus-infected cynomolgus macaques with monoclonal antibodies. Sci Transl Med 4, 138ra181 (2012).
15. A. Marzi et al., Protective efficacy of neutralizing monoclonal antibodies in a nonhuman primate model of Ebola hemorrhagic fever. PLoS One 7, e36192 (2012).
16. X. Qiu et al., Reversion of advanced Ebola virus disease in nonhuman primates with ZMapp. Nature, (2014).
17. G. M. Lyon et al., Clinical care of two patients with Ebola virus disease in the United States. The New England journal of medicine 371, 2402-2409 (2014).
18. A. Frenzel, M. Hust, T. Schirrmann, Expression of recombinant antibodies. Front Immunol 4, 217 (2013).
19. Q. Chen, K. R. Davis, The potential of plants as a system for the development and production of human biologics. F1000Res 5, (2016).
20. R. Strasser et al., Generation of glyco-engineered Nicotiana benthamiana for the production of monoclonal antibodies with a homogeneous human-like N-glycan structure. Plant Biotechnol J 6, 392-402 (2008).
21. J. M. Dias et al., A shared structural solution for neutralizing ebolaviruses. Nat Struct Mol Biol 18, 1424-1427 (2011).
22. J. W. Froude et al., Post-Exposure protection in mice against Sudan virus by a two antibody cocktail. Viruses in press, (2018).
23. A. Z. Wec et al., A “Trojan horse” bispecific-antibody strategy for broad protection against ebolaviruses. Science 354, 350-354 (2016).
24. J. M. Brannan et al., Interferon alpha/beta Receptor-Deficient Mice as a Model for Ebola Virus Disease. J Infect Dis 212 Suppl 2, S282-294 (2015).
25. D. S. Ellis, E. T. Bowen, D. I. Simpson, S. Stamford, Ebola virus: a comparison, at ultrastructural level, of the behaviour of the Sudan and Zaire strains in monkeys. Br J Exp Pathol 59, 584-593 (1978).
26. T. W. Geisbert et al., Recombinant vesicular stomatitis virus vector mediates post exposure protection against Sudan Ebola hemorrhagic fever in nonhuman primates. J Virol 82, 5664-5668 (2008).
27. E. Davidson et al., Mechanism of Binding to Ebola Virus Glycoprotein by the ZMapp, ZMAb, and MB-003 Cocktail Antibodies. J Virol 89, 10982-10992 (2015).
28. C. D. Murin et al., Structures of protective antibodies reveal sites of vulnerability on Ebola virus. Proc Natl Acad Sci USA 111, 17182-17187 (2014).
29. A. Z. Wec et al., Antibodies from a Human Survivor Define Sites of Vulnerability for Broad Protection against Ebolaviruses. Cell 169, 878-890 e815 (2017).
30. X. Zhao et al., Immunization-Elicited Broadly Protective Antibody Reveals Ebolavirus Fusion Loop as a Site of Vulnerability. Cell 169, 891-904 e815 (2017).
31. J. C. Frei et al., Bispecific Antibody Affords Complete Post-Exposure Protection of Mice from Both Ebola (Zaire) and Sudan Viruses. Sci Rep 6, 19193 (2016).
32. F. W. Holtsberg et al., Pan-ebolavirus and Pan-filovirus Mouse Monoclonal Antibodies: Protection against Ebola and Sudan Viruses. J Virol 90, 266-278 (2015).
33. Z. Y. Keck et al., Macaque Monoclonal Antibodies Targeting Novel Conserved Epitopes within Filovirus Glycoprotein. J Virol 90, 279-291 (2015).
34. A. I. Flyak et al., Cross-Reactive and Potent Neutralizing Antibody Responses in Human Survivors of Natural Ebolavirus Infection. Cell 164, 392-405 (2016).
35. Ebola haemorrhagic fever in Sudan, 1976. Report of a WHO/International Study Team. Bull World Health Organ 56, 247-270 (1978).
36. D. Sampey, P. Courville, D. Acree, J. Hausfeld, W. E. Bentley, Enhanced expression of a biosimilar monoclonal antibody with a novel NS0 platform. Biotechnol Prog, (2018).
37. A. C. Wong, R. G. Sandesara, N. Mulherkar, S. P. Whelan, K. Chandran, A forward genetic strategy reveals destabilizing mutations in the Ebolavirus glycoprotein that alter its protease dependence during cell entry. J Virol 84, 163-175 (2010).

What is claimed is:

1. A composition for the treatment of Sudan virus (SUDV) infection in a patient, the composition comprising a therapeutically effective combination of:
(i) a first monoclonal antibody or an antigen-binding fragment thereof which binds to the base of SUDV glycoprotein (GP) trimer, optionally which binds to at least one amino acid within SUDV glycoprotein 1 (GP1) and at least one amino acid within SUDV glycoprotein 2 (GP2); and
(ii) a second monoclonal antibody or an antigen-binding fragment thereof which binds to the glycan cap of SUDV GP.

2. The composition of claim 1, wherein the second monoclonal antibody or an antigen-binding fragment thereof comprises:
(a) a VH comprising a CDR1, a CDR2, and a CDR3 comprising the amino acid sequence of SEQ ID NOs: 27, 28, and 29, respectively; and
(b) a VL comprising a CDR1, a CDR2, and a CDR3 comprising the amino acid sequence of SEQ ID NOs: 24, 25, and 26, respectively.

3. The composition of claim 2, wherein:
in (a), the VH comprises an amino acid sequence with at least 80%, 85%, 90%, 91%, 92%, 93%, 94%, 95%, 96%, 97%, 98% or 99% sequence identity to the amino acid sequence of SEQ ID NO: 13; and
in (b), the VL comprises an amino acid sequence with at least 80%, 85%, 90%, 91%, 92%, 93%, 94%, 95%, 96%, 97%, 98% or 99% sequence identity to the amino acid sequence of SEQ ID NO: 14.

4. The composition of claim 2, wherein:
in (a), the VH comprises an amino acid sequence of SEQ ID NO: 13; and
in (b), the VL comprises an amino acid sequence of SEQ ID NO: 14.

5. The composition of claim 1, wherein the first monoclonal antibody or an antigen-binding fragment thereof comprises:
(I) (I-a) a heavy chain variable region (VH) comprising a complementarity-determining region 1 (CDR1), a complementarity-determining region 2 (CDR2), and a complementarity-determining region 3 (CDR3) comprising the amino acid sequence of SEQ ID NOs: 38, 39, and 40, respectively, and (I-b) a light chain variable region (VL) comprising a CDR1, a CDR2, and a CDR3 comprising the amino acid sequence of SEQ ID NOs: 41, 42, and 43, respectively; or
(II) (II-a) a VH comprising a CDR1, a CDR2, and a CDR3 comprising the amino acid sequence of SEQ ID NOs: 30, 31, and 32, respectively, and (II-b) a VL comprising a CDR1, a CDR2, and a CDR3 comprising the amino acid sequence of SEQ ID NOs: 33, 34, and 35, respectively.

6. The composition of claim 5, wherein:
in (I), (I-a) the VH comprises an amino acid sequence with at least 80%, 85%, 90%, 91%, 92%, 93%, 94%, 95%, 96%, 97%, 98% or 99% sequence identity to the amino acid sequence of SEQ ID NO: 36 and (I-b) the VL comprises an amino acid sequence with at least 80%, 85%, 90%, 91%, 92%, 93%, 94%, 95%, 96%, 97%, 98% or 99% sequence identity to the amino acid sequence of SEQ ID NO: 37; or
in (II), (II-a) the VH comprises an amino acid sequence with at least 80%, 85%, 90%, 91%, 92%, 93%, 94%, 95%, 96%, 97%, 98% or 99% sequence identity to the amino acid sequence of SEQ ID NO: 5 and (II-b) the VL comprises an amino acid sequence with at least 80%, 85%, 90%, 91%, 92%, 93%, 94%, 95%, 96%, 97%, 98% or 99% sequence identity to the amino acid sequence of SEQ ID NO: 6.

7. The composition of claim 5, wherein:
in (I), (I-a) the VH comprises an amino acid sequence of SEQ ID NO: 36 and (I-b) the VL comprises an amino acid sequence of SEQ ID NO: 37; or
in (II), (II-a) the VH comprises an amino acid sequence of SEQ ID NO: 5 and (II-b) the VL comprises an amino acid sequence of SEQ ID NO: 6.

8. The composition of claim 5, wherein:
in (I), (I-a) the VH is a humanized variant of a VH comprising an amino acid sequence of SEQ ID NO: 5 and (I-b) the VL is a humanized variant of a VL comprising an amino acid sequence of SEQ ID NO: 6.

9. The composition of claim 1, wherein
the first monoclonal antibody or an antigen-binding fragment thereof comprises:
(a) a VH comprising an amino acid sequence of SEQ ID NO: 36; and
(b) a VL comprises an amino acid sequence of SEQ ID NO: 37,
and wherein the second monoclonal antibody or an antigen-binding fragment thereof comprises:
(a) a VH comprising an amino acid sequence of SEQ ID NO: 13; and
(b) a VL comprising an amino acid sequence of SEQ ID NO: 14.

10. The composition of claim 1, further comprising: a pharmaceutically acceptable excipient or carrier.

11. The composition of claim 1, wherein the patient is a human.

12. A method of treating Sudan virus infection in a patient, the method comprising:
(i) identifying a patient in need of Sudan virus treatment; and
(ii) administering to the patient a therapeutically effective amount of a composition of claim 1.

13. The method of claim 12, wherein the patient is a human.

14. A monoclonal antibody or antigen-binding fragment thereof, which comprises:
(a) a VH comprising a CDR1, a CDR2, and a CDR3 comprising the amino acid sequence of SEQ ID NOs: 27, 28, and 29, respectively; and
(b) a VL comprising a CDR1, a CDR2, and a CDR3 comprising the amino acid sequence of SEQ ID NOs: 24, 25, and 26, respectively.

15. The monoclonal antibody or antigen-binding fragment thereof of claim 14, wherein:
in (a), the VH comprises an amino acid sequence with at least 80%, 85%, 90%, 91%, 92%, 93%, 94%, 95%, 96%, 97%, 98% or 99% sequence identity to the amino acid sequence of SEQ ID NO: 13; and
in (b), the VL comprises an amino acid sequence with at least 80%, 85%, 90%, 91%, 92%, 93%, 94%, 95%, 96%, 97%, 98% or 99% sequence identity to the amino acid sequence of SEQ ID NO: 14.

16. The monoclonal antibody or antigen-binding fragment thereof of claim 14, wherein:
in (a), the VH comprises an amino acid sequence of SEQ ID NO: 13; and
in (b), the VL comprises an amino acid sequence of SEQ ID NO: 14.

17. The monoclonal antibody or antigen-binding fragment thereof of claim 14, wherein the sequences or part of the sequences are contained in Fab, Fab′, F(ab′)₂, Fv, CDR fragments, single-chain antibodies (e.g. scFv), humanized antibodies, chimeric antibodies, or bispecific antibodies.

18. A single chain variable fragment (scFv) which binds to the glycan cap of SUDV GP, wherein the scFv comprises:
(A) a VH, a linker, and a VL, in the direction from the N-terminus to the C-terminus; or
(B) a VL, a linker, and a VH, in the direction from the N-terminus to the C-terminus,
wherein:
(a) the VH comprises a CDR1, a CDR2, and a CDR3 comprising the amino acid sequence of SEQ ID NOs: 27, 28, and 29, respectively; and
(b) a VL comprises a CDR1, a CDR2, and a CDR3 comprising the amino acid sequence of SEQ ID NOs: 24, 25, and 26, respectively.

19. The scFv of claim 18, wherein:
(a) the VH comprises an amino acid sequence with at least 80%, 85%, 90%, 91%, 92%, 93%, 94%, 95%, 96%, 97%, 98% or 99% sequence identity to the amino acid sequence of SEQ ID NO: 13; and
(b) the VL comprises an amino acid sequence with at least 80%, 85%, 90%, 91%, 92%, 93%, 94%, 95%, 96%, 97%, 98% or 99% sequence identity to the amino acid sequence of SEQ ID NO: 14.

20. The scFv of claim 18, wherein:
(a) the VH comprises an amino acid sequence of SEQ ID NO: 13; and
(b) the VL comprises an amino acid sequence of SEQ ID NO: 14.

21. The scFv of claim 18 comprising the amino acid sequence of SEQ ID NO. 23.

22. The monoclonal antibody or antigen-binding fragment thereof of claim 14, which is manufactured in:
(i) a plant cell, optionally wherein the plant is Nicotiana benthamiana; or
(ii) a mammalian cell, optionally NS0 cell, CHO cell, 293 cell, or hybridoma.

23. A method of treating Sudan virus infection in a patient, the method comprising:
administering to the patient a therapeutically effective amount of a composition comprising the monoclonal antibody or antigen-binding fragment thereof of claim 14.

24. A host cell comprising a nucleic acid sequence encoding the monoclonal antibody or antigen-binding fragment thereof of claim 14, optionally wherein the host cell is:
(i) a plant cell, optionally wherein the plant is Nicotiana benthamiana; or
(ii) a mammalian cell, optionally an NS0 cell, CHO cell, 293 cell, or hybridoma.

25. (canceled)

26. A composition for the treatment of Sudan virus (SUDV) infection in a patient, the composition comprising a therapeutically effective amount of a monoclonal antibody or an antigen-binding fragment thereof which binds to the glycan cap of SUDV GP.

27. The composition of claim 26, wherein the monoclonal antibody or an antigen-binding fragment thereof comprises:
(a) a VH comprising a CDR1, a CDR2, and a CDR3 comprising the amino acid sequence of SEQ ID NOs: 27, 28, and 29, respectively; and
(b) a VL comprising a CDR1, a CDR2, and a CDR3 comprising the amino acid sequence of SEQ ID NOs: 24, 25, and 26, respectively.

28. The composition of claim 27, wherein:
in (a), the VH comprises an amino acid sequence with at least 80%, 85%, 90%, 91%, 92%, 93%, 94%, 95%, 96%, 97%, 98% or 99% sequence identity to the amino acid sequence of SEQ ID NO: 13; and
in (b), the VL comprises an amino acid sequence with at least 80%, 85%, 90%, 91%, 92%, 93%, 94%, 95%, 96%, 97%, 98% or 99% sequence identity to the amino acid sequence of SEQ ID NO: 14.

29. The composition of claim 27, wherein:
in (a), the VH comprises an amino acid sequence of SEQ ID NO: 13; and
in (b), the VL comprises an amino acid sequence of SEQ ID NO: 14.

30. A method of treating Sudan virus infection in a patient, the method comprising:
administering to the patient a therapeutically effective amount of a composition of claim 26.

31. The scFv of claim 18, which is manufactured in:
(i) a plant cell, optionally wherein the plant is Nicotiana benthamiana; or
(ii) a mammalian cell, optionally NS0 cell, CHO cell, 293 cell, or hybridoma.

32. A method of treating Sudan virus infection in a patient, the method comprising:
administering to the patient a therapeutically effective amount of a composition comprising the scFv of claim 18.

33. A host cell comprising a nucleic acid sequence encoding the scFv of claim 18, optionally wherein the host cell is:
(i) a plant cell, optionally wherein the plant is Nicotiana benthamiana; or
(ii) a mammalian cell, optionally an NS0 cell, CHO cell, 293 cell, or hybridoma.
